(19) United States
(12) Patent Application Publication
(10) Pub. No.: US 2024/0298926 A1
(43) Pub. Date: Sep. 12, 2024

(54) GAIT EVALUATION DEVICE, GAIT EVALUATION METHOD, GAIT MEASUREMENT SYSTEM AND RECORDING MEDIUM

(71) Applicant: NEC CORPORATION, Minato-ku, Tokyo (JP)

(72) Inventors: Chenhui Huang, Tokyo (JP); Fumiyuki Nihey, Tokyo (JP)

(73) Assignee: NEC CORPORATION, Minato-ku, Tokyo (JP)

(21) Appl. No.: 18/575,116

(22) PCT Filed: Jul. 12, 2021
(86) PCT No.: PCT/JP2021/026069

§ 371 Date: Dec. 28, 2023

(51) Int. Cl. A61B 5/11; A61B 5/00

(52) U.S. Cl. CPC A61B 5/112; A61B 5/1114; A61B 5/4519; A61B 5/6807; A61B 2562/0219

(57) ABSTRACT

A gait evaluation device that includes an identification unit that, based on sensor data regarding movement of a foot, identifies a walking session in which stable walking is performed, a waveform processing unit that, from time series data of the sensor data measured for a same walking session, extracts, for each gait cycle, a target waveform which is included in an evaluation target segment of dynamic stability of gait, and a stability evaluation unit that evaluates the dynamic stability of gait in response to a transition of similarity of the target waveform extracted in each gait cycle, and outputs an evaluation result of the dynamic stability of gait.

### TECHNICAL FIELD

The present disclosure relates to a gait evaluation device or the like that evaluates dynamic stability in gait.

### BACKGROUND ART

With increasing interest in healthcare that performs physical condition management, a service that measures features included in a walking pattern (also referred to as gait) and provides information relevant to the gait to a user has attracted attention. For example, a measurement device including an inertial sensor is mounted on footwear such as shoes, and a device for analyzing a gait of a user has been developed. If the dynamic stability of gait can be evaluated by using data (also referred to as sensor data) measured by the measurement device along with walking of the user wearing the footwear on which the measurement device is mounted, the risk of falling of the user or the like can be predicted. The dynamic stability of gait is an index related to walking ability.

PTL 1 discloses a determination device that performs walking determination using sensor data measured by a measurement device mounted on footwear. The device of PTL 1 determines the walking state according to the values of the acceleration in the gravity direction and the acceleration in the traveling direction, and switches modes in the walking measurement. The device of PTL 1 switches from a power saving mode to a discrimination mode when the value of the acceleration in the gravity direction exceeds a first threshold. The device of PTL 1 switches from the discrimination mode to a gait measurement mode when the value of the acceleration in the traveling direction exceeds a second threshold in the discrimination mode. The device of PTL 1 detects a change tendency of a peak value using log data of the peak value of the acceleration in the traveling direction in the discrimination mode. The device of PTL 1 changes the second threshold based on the detected change tendency of the peak value.

PTL 2 discloses a gait motion analyzing device that analyzes a gait motion of a subject based on a measurement result of a gait motion measurement unit such as motion capture or a foot pressure distribution measurement device. The device of PTL 2 determines whether the subject is walking steadily from the measurement result of the gait motion measurement unit. For example, the device of PTL 2 determines whether steady walking is performed based on a fluctuation range (variation) of a predetermined parameter such as a stride and an upper arm angle in a measurement target period during walking. For example, the device of PTL 2 determines that the subject walks in a steady state when a predetermined parameter in the measurement target period converges within a certain width, and determines that the subject walks in an abnormal state when the predetermined parameter does not converge within the certain width even after a lapse of a predetermined time.

PTL 3 discloses a gait estimation device that estimates a gait of a user. The device of PTL 3 calculates first to third feature amounts regarding each of power, pace, and body balance during walking using acceleration data measured by an acceleration sensor. The device of PTL 3 calculates a first to third feature amounts based on an acceleration norm waveform in a certain segment of acceleration data.

### CITATION LIST

### Patent Literature

PTL 1: WO 2020/230282 A1
PTL 2: JP 2009-125270 A
PTL 3: JP 6564711 B2

### SUMMARY OF INVENTION

### Technical Problem

According to the method of PTL 1, by changing the second threshold based on the change tendency of the peak value of the acceleration in the traveling direction in the determination mode, it is possible to achieve both high efficiency and low power consumption of the gait measurement while flexibly responding to the change in the walking state. According to the method of PTL 2, it is possible to determine steady walking or abnormal walking based on a variation in a predetermined parameter in a measurement target period. However, in the methods of PTLs 1 and 2, it is not possible to determine the dynamic stability of gait.

In the method of PTL 3, the third feature amount regarding the body balance (dynamic stability) at the time of walking is calculated based on the autocorrelation coefficient of the acceleration norm waveform in the segment for each certain segment. That is, in the method of PTL 3, the body balance ability is evaluated based on the similarity of the acceleration waveform for each step. In the method of PTL 3, since the body balance ability is evaluated based on the similarity of the acceleration waveform for each step, physical factors that affect the dynamic stability during walking are not reflected. In the method of PTL 3, the third feature amount is easily affected by an accidental variation in gait. Therefore, in the method of PTL 3, it is difficult to improve the evaluation ability and accuracy of the dynamic stability of gait.

An object of the present disclosure is to provide a gait evaluation device and the like that can accurately evaluate dynamic stability of gait.

### Solution to Problem

A gait evaluation device according to an aspect of the present disclosure includes an identification unit that, based on sensor data regarding movement of a foot, identifies a walking session in which stable walking is performed, a waveform processing unit that, from time series data of the sensor data measured for a same walking session, extracts, for each gait cycle, a target waveform which is included in an evaluation target segment of dynamic stability of gait, and a stability evaluation unit that evaluates the dynamic stability of gait in response to a transition of similarity of the target waveform extracted in each gait cycle, and outputs an evaluation result of the dynamic stability of gait.

In a gait evaluation method according to an aspect of the present disclosure, a computer executes: identifying a walking session in which stable walking is performed based on sensor data regarding movement of a foot, extracting a target waveform included in an evaluation target segment of dynamic stability of gait from time series data of the sensor data measured for a same walking session for each gait cycle, evaluating the dynamic stability of gait in response to a transition of similarity of the target waveform extracted for each gait cycle, and outputting an evaluation result of the dynamic stability of gait.

A program according to an aspect of the present disclosure causes a computer to execute a process of identifying, based on sensor data regarding movement of a foot, a walking session in which stable walking is performed, a process of extracting, from time series data of the sensor data measured for a same walking session, for each gait cycle, a target waveform which is included in an evaluation target segment of dynamic stability of gait; evaluating the dynamic stability of gait in response to a transition of similarity of the target waveform extracted in each gait cycle, and a process of outputting an evaluation result of the dynamic stability of gait.

### Advantageous Effects of Invention

According to the present disclosure, it is possible to provide a gait evaluation device and the like that can accurately evaluate dynamic stability of gait.

### BRIEF DESCRIPTION OF DRAWINGS

FIG. 1 is a block diagram illustrating an example of a configuration of a gait measurement system according to a first example embodiment.

FIG. 2 is a conceptual diagram illustrating an arrangement example of a measurement device of the gait measurement system according to the first example embodiment.

FIG. 3 is a conceptual diagram for explaining a coordinate system set in the measurement device of the gait measurement system according to the first example embodiment.

FIG. 4 is a conceptual diagram for explaining an example of a gait cycle used in the description of the gait measurement system according to the first example embodiment.

FIG. 5 is a conceptual diagram for describing a transition of similarity of a target waveform to be evaluated by the gait evaluation device of the gait measurement system according to the first example embodiment.

FIG. 6 is a block diagram illustrating an example of a configuration of the measurement device of the measurement system according to the first example embodiment.

FIG. 7 is a block diagram illustrating an example of a configuration of the gait evaluation device of the measurement system according to the first example embodiment.

FIG. 8 is an example of a transition of similarity of a target waveform to be evaluated by the gait evaluation device of the gait measurement system according to the first example embodiment.

FIG. 9 is another example of a transition of similarity of a target waveform to be evaluated by the gait evaluation device of the gait measurement system according to the first example embodiment.

FIG. 10 is a conceptual diagram illustrating an example of a usage scene of the gait measurement system according to the first example embodiment.

FIG. 11 is a flowchart for explaining an example of an operation of the gait evaluation device of the gait measurement system according to the first example embodiment.

FIG. 12 is a flowchart for explaining an example of waveform generation processing by the gait evaluation device of the gait measurement system according to the first example embodiment.

FIG. 13 is a flowchart for explaining an example of dynamic stability evaluation processing by the gait evaluation device of the gait measurement system according to the first example embodiment.

FIG. 14 is a block diagram illustrating an example of a configuration of a gait measurement system according to a second example embodiment.

FIG. 15 is a block diagram illustrating an example of a configuration of the gait evaluation device of the measurement system according to the second example embodiment.

FIG. 16 is a conceptual diagram illustrating an example of a similarity matrix generated by the gait evaluation device of the measurement system according to the second example embodiment.

FIG. 17 is a conceptual diagram illustrating another example of a similarity matrix generated by the gait evaluation device of the measurement system according to the second example embodiment.

FIG. 18 is a conceptual diagram illustrating an example of a usage scene of the gait measurement system according to the second example embodiment.

FIG. 19 is a flowchart for explaining an example of an operation of the gait evaluation device of the gait measurement system according to the second example embodiment.

FIG. 20 is a flowchart for explaining an example of waveform generation processing by the gait evaluation device of the gait measurement system according to the second example embodiment.

FIG. 21 is a flowchart for explaining an example of dynamic stability evaluation processing by the gait evaluation device of the gait measurement system according to the second example embodiment.

FIG. 22 is a block diagram illustrating an example of a configuration of a gait evaluation device according to a third example embodiment.

FIG. 23 is a conceptual diagram illustrating an example of a hardware configuration that executes processing according to each example embodiment.

### EXAMPLE EMBODIMENT

Hereinafter, example embodiments of the present invention will be described with reference to the drawings. However, the example embodiments described below may be technically limited for carrying out the present invention, but the scope of the invention is not limited to the following. In all the drawings used in the following description of the example embodiment, the same reference numerals are given to the same parts unless there is a particular reason. In the following example embodiments, repeated description of similar configurations and operations may be omitted.

### First Example Embodiment

First, an example of a configuration of a gait measurement system according to a first example embodiment will be described with reference to the drawings. The gait measurement system according to the present example embodiment measures a physical quantity (sensor data) regarding a movement of a foot by a measurement device installed on footwear worn by a user. The measurement device includes an acceleration sensor and an angular velocity sensor. For example, the physical quantity regarding the movement of the foot includes accelerations in three axial directions (also referred to as spatial accelerations) measured by the acceleration sensor and angular velocities around three axes (also referred to as spatial angular velocities) measured by the angular velocity sensor. The gait measurement system of the present example embodiment evaluates the dynamic stability of gait using the measured sensor data.

### (Configuration)

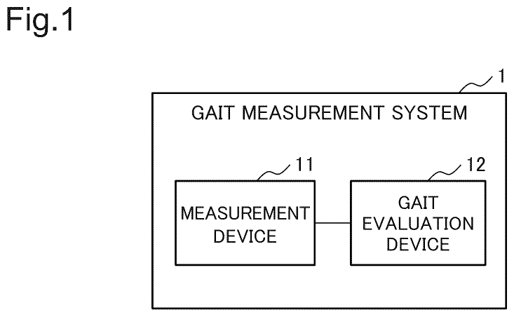

FIG. 1 is a block diagram illustrating a configuration of a gait measurement system 1 according to the present example embodiment. The gait measurement system 1 includes a measurement device 11 and a gait evaluation device 12. The gait evaluation device 12 may be connected to the measurement device 11 in a wireless manner. The measurement device 11 and the gait evaluation device 12 may be configured by a single device. The gait measurement system 1 may include only the gait evaluation device 12 except for the measurement device 11.

The measurement device 11 is installed on the foot portion. For example, the measurement device 11 is installed in a footwear such as shoes. For example, the measurement device 11 is disposed at a position on the back side of the arch of foot. The measurement device 11 includes an acceleration sensor and an angular velocity sensor. The measurement device 11 measures acceleration measured by the acceleration sensor (also referred to as spatial acceleration) and an angular velocity measured by the angular velocity sensor (also referred to as spatial angular velocity) as physical quantities regarding the movement of the foot of the user wearing the footwear. The physical quantity regarding the movement of the foot measured by the measurement device 11 includes a velocity, an angle, and a position (trajectory) calculated by integrating the acceleration and the angular velocity. The measurement device 11 converts the measured physical quantity into digital data (also referred to as sensor data). The measurement device 11 transmits the converted sensor data to the gait evaluation device 12. For example, the sensor data includes a time stamp relevant to a time at which the sensor data is acquired. The time stamp is a time-series number assigned to the sensor data. For example, the measurement device 11 is connected to the gait evaluation device 12 via a mobile terminal (not illustrated) carried by the user.

A mobile terminal (not illustrated) is a communication device that can be carried by a user. For example, the mobile terminal is a portable communication device having a communication function, such as a smartphone, a smart watch, or a mobile phone. The mobile terminal receives, from the measurement device 11, sensor data regarding the movement of the foot of the user. The mobile terminal transmits the received sensor data to a server, a cloud, or the like on which the gait evaluation device 12 is mounted. The functions of the gait evaluation device 12 may be achieved by application software or the like installed in the mobile terminal. In this case, the mobile terminal processes the received sensor data by application software or the like installed therein.

The measurement device 11 is achieved by, for example, an inertial measurement device including an acceleration sensor and an angular velocity sensor. An example of the inertial measurement device is an inertial measurement unit (IMU). The IMU includes an acceleration sensor that measures accelerations in three axial directions and an angular velocity sensor that measures angular velocities around three axes. The measurement device 11 may be achieved by an inertial measurement device such as a vertical gyro (VG) or an attitude heading (AHRS). The measurement device 11 may be achieved by a Global Positioning System/Inertial Navigation System (GPS/INS).

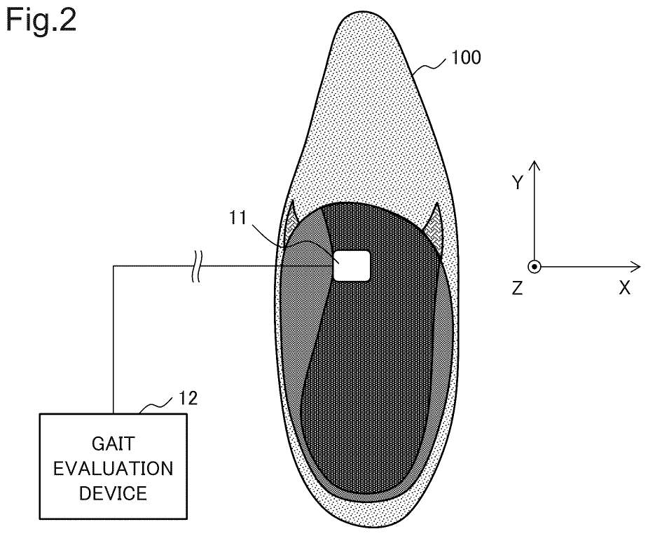

FIG. 2 is a conceptual diagram illustrating an example of disposed the measurement device 11 in a shoe 100. In the example of FIG. 2, the measurement device 11 is disposed at a position relevant to the back side of the arch of foot. For example, the measurement device 11 is disposed in an insole inserted into the shoe 100. For example, the measurement device 11 is disposed on the bottom surface of the shoe 100. For example, the measurement device 11 may be embedded in the main body of the shoe 100. The measurement device 11 may be detachable from the shoe 100 or may not be detachable from the shoe 100. The measurement device 11 may be disposed at a position other than the back side of the arch of foot as long as the sensor data regarding the movement of the foot can be acquired. The measurement device 11 may be disposed on a sock worn by the user or a decorative article such as an anklet worn by the user. The measurement device 11 may be directly attached to the foot or may be embedded in the foot. FIG. 2 illustrates an example in which the measurement device 11 is disposed on the shoe 100 on the right foot side, but the measurement device 11 may be disposed on the shoe 100 for both feet. If the measurement device 11 is disposed on the shoe 100 for both feet, the gait can be measured based on the movement of the foot for both feet.

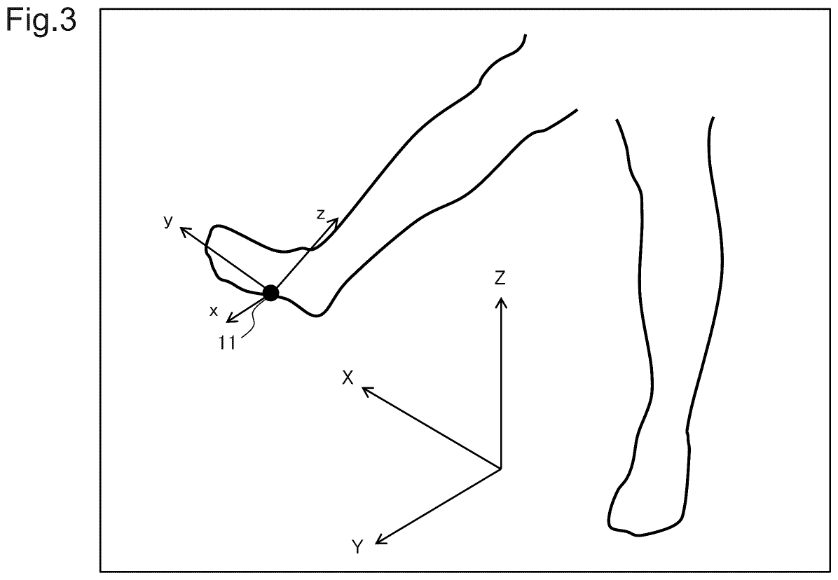

FIG. 3 is a conceptual diagram for explaining a local coordinate system (x axis, y axis, z axis) set in the measurement device 11 and a world coordinate system (X axis, Y axis, Z axis) set with respect to the ground in a case where the measurement device 11 is installed on the back side of the arch of foot. In the world coordinate system (X axis, Y axis, Z axis), in a state where the user is standing upright, a lateral direction of the user is set to an X-axis direction (rightward direction is positive), a front direction of the user (traveling direction) is set to a Y-axis direction (forward direction is positive), and a gravity direction is set to a Z-axis direction (vertically upward direction is positive). In the present example embodiment, the local coordinate system including an x direction, a y direction, and a z direction based on the measurement device 11 is set. The directions of the axes of the local coordinate system and the world coordinate system are not limited to the directions in FIG. 3, and may be any directions as long as they can be converted from each other.

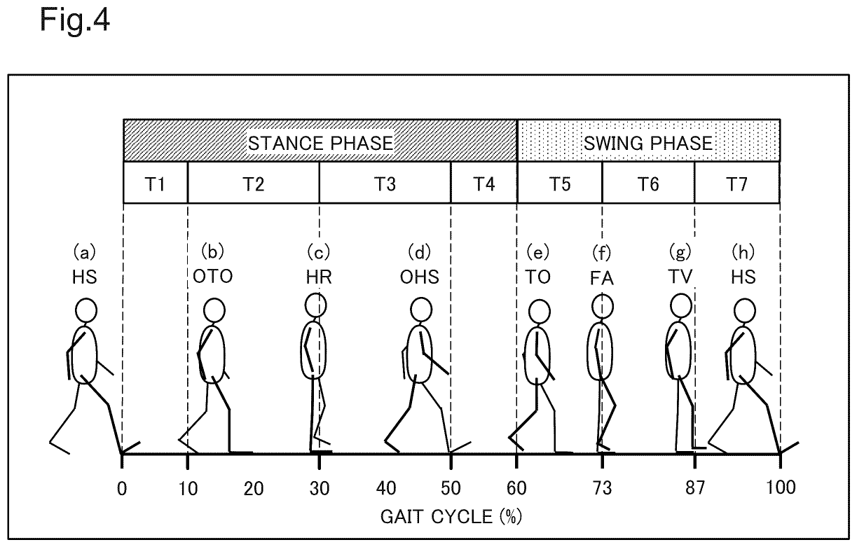

FIG. 4 is a conceptual diagram for explaining one gait cycle based on the right foot. FIG. 4 illustrates one gait cycle of the right foot with a time point at which the heel of the right foot lands on the ground as a start point and a time point at which the heel of the right foot next lands on the ground as an end point. The gait cycle in FIG. 4 is normalized with one gait cycle of the right foot as 0 to 100%. The timing of each % of the gait cycle is also referred to as a gait phase. The one gait cycle of one foot is roughly divided into a stance phase in which at least a part of the back side of the foot is in contact with the ground and a swing phase in which the back side of the foot is separated from the ground. In general, in the gait cycle, the stance phase occupies 60%, and the swing phase occupies 40%. For example, the gait cycle is normalized such that the stance phase occupies 60% and the swing phase occupies 40%. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. The segments such as the initial stance period T1, the mid-stance period T2, the terminal stance period T3, the pre-swing period T4, the initial swing period T5, the mid-swing period T6, and the terminal swing period T7 are also referred to as gait periods. Segments such as the stance phase and the swing phase are also included in the gait period. The gait waveform for one gait cycle may not have a time point when the heel lands on the ground as the start point. For example, the gait waveform for one gait cycle may have a start point and an end point at which the heel rises.

In one gait cycle, a plurality of events (referred to as gait events) occur. FIG. 4(a) represents an event (heel strike) in which the heel of the right foot lands on the ground (HS: Heel Strike). FIG. 4(b) illustrates an event (opposite toe off) in which the toe of the left foot is separated from the ground while the sole of the right foot is in contact with the ground (OTO: Opposite Toe Off). FIG. 4(c) illustrates an event (heel rise) in which the heel of the right foot is lifted while the sole of the right foot is in contact with the ground (HR: Heel Rise). FIG. 4(d) is an event (opposite heel strike) in which the heel of the left foot is grounded (OHS: Opposite Heel Strike). FIG. 4(e) illustrates an event (toe off) in which the toe of the right foot is separated from the ground while the sole of the left foot is in contact with the ground (TO: Toe Off). FIG. 4(f) illustrates an event (foot adjacent) in which the left foot and the right foot cross each other while the sole of the left foot is in contact with the ground (FA: Foot Adjacent). FIG. 4(g) illustrates an event (tibia vertical) in which the tibia of the right foot is substantially perpendicular to the ground while the sole of the left foot is in contact with the ground (TV: Tibia Vertical). FIG. 4(h) illustrates an event (heel strike) in which the heel of the right foot is grounded (HS: Heel Strike). FIG. 4(h) is relevant to the end point of the gait cycle starting from FIG. 4(a), and relevant to the start point of the next gait cycle. The timing at which the gait event appears differs depending on a personal physical condition, and a gait state, and thus does not completely coincide with the assumed gait cycle.

The gait period and the gait event are associated as follows. The initial stance period T1 is a period from the heel strike HS to the opposite toe off OTO. The mid-stance period T2 is a period from the opposite toe off OTO to the heel rise HR. The terminal stance period T3 is a period from the heel rise HR to the opposite heel strike OHS. The pre-swing period T4 is a period from the opposite heel strike OHS to the toe off TO. The initial swing period T5 is a period from the toe off TO to the foot adjacent FA. The mid-swing period T6 is a period from the foot adjacent FA to the tibia vertical TV. The terminal swing period T7 is a period from the tibia vertical TV to the heel strike HS.

The gait evaluation device 12 receives sensor data from the measurement device 11. The gait evaluation device 12 detects the start of stable walking based on the received sensor data. For example, the gait evaluation device 12 detects the start of stable walking according to a relationship between a peak value of the acceleration in the traveling direction (acceleration in the Y direction) and a threshold (also referred to as a first threshold). For example, the gait evaluation device 12 can be configured to detect the start of stable walking when the peak value of the acceleration in the traveling direction (acceleration in the Y direction) exceeds the first threshold three times. The gait evaluation device 12 evaluates the dynamic stability of gait in a segment (also referred to as a walking session) from a time point at which the start of stable walking is detected to a time point at which the end of stable walking is detected. A walking session is also referred to as a walking bout.

Upon detecting the start of stable walking, the gait evaluation device 12 generates time series data of sensor data measured by the measurement device 11 for the same walking session. The gait evaluation device 12 measures the number of steps of the user in accordance with the generation of the time series data of the sensor data. The gait evaluation device 12 cuts out a waveform from the time series data of the sensor data in accordance with the gait cycle. For example, the gait evaluation device 12 cuts out a waveform for one gait cycle from time series data of sensor data for one gait cycle. For example, the gait evaluation device 12 may cut out a waveform of one step from time series data of sensor data of one step. For example, the gait evaluation device 12 may cut out a waveform for one stride from time series data of sensor data for one stride. The gait evaluation device 12 normalizes a horizontal axis (time) of the cut-out waveform to a gait cycle of 0 to 100%. The gait evaluation device 12 normalizes a vertical axis (intensity) of the cut-out waveform. For example, the gait evaluation device 12 normalizes the vertical axis (intensity) of the cut-out waveform with the maximum intensity as a reference.

The gait evaluation device 12 extracts a waveform (also referred to as a target waveform) to be evaluated for the dynamic stability of gait from a normalized waveform (also referred to as a gait waveform) for one gait cycle. For example, the gait evaluation device 12 extracts a waveform included in the period of the swing phase as the target waveform. The gait evaluation device 12 extracts target waveforms in all gait cycles in one walking session. The gait evaluation device 12 evaluates dynamic stability of gait based on a change in similarity of a target waveform in a walking session. For example, the gait evaluation device 12 tracks the similarity of the short-term, mid-term, and long-term target waveforms in the same walking session.

The gait evaluation device 12 calculates similarity between a target waveform (also referred to as a reference target waveform) extracted from a gait waveform of a gait cycle as a reference and a target waveform extracted from gait waveforms of other gait cycles in the same walking session. For example, the gait evaluation device 12 calculates similarity between a target waveform extracted from a gait waveform in the first gait cycle and a target waveform extracted from a gait waveform in a series of subsequent gait cycles in the same walking session. For example, the gait evaluation device 12 may calculate similarity between a target waveform extracted from a gait waveform after several gait cycles and a target waveform extracted from a gait waveform in a series of subsequent gait cycles in the same walking session. For example, the gait evaluation device 12 may calculate similarity between a representative value of a target waveform extracted from a gait waveform for several gait cycles and a representative value of a target waveform extracted from a gait waveform of a series of subsequent gait cycles in the same walking session. The gait evaluation device 12 may calculate similarity for each stride or the number of steps. A method of calculating the similarity by the gait evaluation device 12 will be described later.

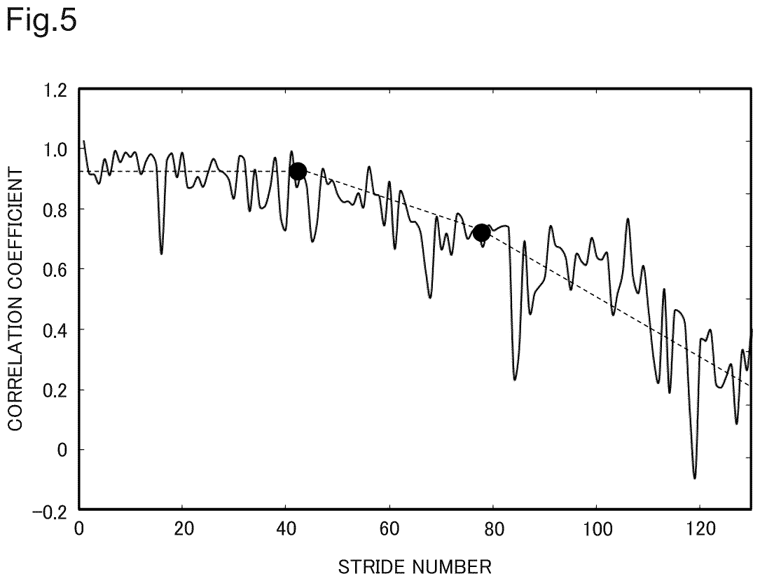

FIG. 5 is a graph illustrating a change in similarity (correlation coefficient) between a target waveform (also referred to as a reference target waveform) extracted from a gait waveform in the first gait cycle and a target waveform extracted from a gait waveform in a subsequent series of gait cycles. In the example of FIG. 5, the target waveform extracted from the gait waveform in the first gait cycle is set as the reference target waveform. For example, the target waveform extracted from the gait waveform after several gait cycles from the start of walking may be set as the reference target waveform. For example, a waveform obtained by averaging a plurality of target waveforms extracted from a gait waveform after several gait cycles from the start of walking may be used as the reference target waveform. The horizontal axis of the graph of FIG. 5 is the stride number. The vertical axis of the graph of FIG. 5 is a correlation coefficient between the target waveform extracted from the gait waveform of the first step and the target waveform extracted for each number of steps. In FIG. 5, the baseline of the similarity of the target waveform is indicated by a broken line. Since the change in the similarity of the target waveform varies greatly every step, it is difficult to verify the similarity every step. However, the change in the similarity of the target waveform over a long period of time tends to be related to the dynamic stability of gait. For example, the similarity of the target waveform tends to decrease as the stride number increases. The gait evaluation device 12 evaluates the dynamic stability of gait based on a change in similarity of the target waveform over a long period of time.

As illustrated in FIG. 5, the decreasing rate of the similarity of the target waveform (the slope of the baseline) tends to increase as the stride number increases. In a period of about 0 to 40 steps (initial stage of walking), the inclination of the baseline is substantially 0. That is, it is difficult to verify a change in similarity at the initial stage of walking. In a period of about 40 to 80 steps, a negative slope is seen in the baseline. In a period exceeding 80 steps, the absolute value of the baseline slope increases. In this manner, the decreasing tendency of the similarity of the target waveform changes according to the progress of walking. For example, the decreasing tendency of the similarity of the target waveform is mainly caused by muscle fatigue, and depends on the attribute and physical condition of an individual. For example, a decrease in similarity of the target waveform according to the progress of walking tends to be more significant in the elderly group than in the young group. For example, the more fatigued the subject is, the more the similarity of the target waveform tends to decrease in accordance with the progress of walking.

The gait evaluation device 12 evaluates the dynamic stability of gait of the user in response to the transition of the similarity of the target waveform accompanying the walking of the user. For example, the gait evaluation device 12 evaluates the dynamic stability of gait according to a decreasing tendency of the similarity of the target waveform. For example, in a case where the decreasing rate of the similarity of the target waveform falls below a predetermined threshold, the gait evaluation device 12 determines that the dynamic stability of gait has decreased. For example, in a case where the decreasing rate of the similarity of the target waveform falls below a predetermined threshold for a predetermined period, the gait evaluation device 12 determines that the dynamic stability of gait has decreased. For example, in a case where the absolute value of the baseline slope of the similarity of the target waveform exceeds a predetermined value, the gait evaluation device 12 determines that the dynamic stability of gait has decreased. For example, in a case where the absolute value of the baseline slope of the similarity of the target waveform rapidly increases, the gait evaluation device 12 determines that the dynamic stability of gait has decreased. Details of the evaluation of the dynamic stability of gait by the gait evaluation device 12 will be described later.

For example, the gait evaluation device 12 evaluates the dynamic stability of gait according to a change in similarity with the progress of walking. For example, the gait evaluation device 12 evaluates the dynamic stability of gait according to the difference between the representative value of the similarity between the reference target waveform and the target waveform in the first stage where the number of steps is less than the predetermined number of steps and the representative value of the similarity between the reference target waveform and the target waveform in the second stage where the number of steps is equal to or greater than the predetermined number of steps. For example, the gait evaluation device 12 evaluates the dynamic stability of gait by comparing representative values such as an average value, a mode value, and a median value. For example, the gait evaluation device 12 evaluates the dynamic stability of gait by comparing average values such as an arithmetic mean, a geometric mean, a harmonic mean, and a logarithmic mean.

For example, the gait evaluation device 12 evaluates the dynamic stability of gait according to the difference between the representative value of the similarity between the reference target waveform and the target waveform in the first stage where the number of steps is less than the predetermined number of steps and the representative value of the similarity between the reference target waveform and the target waveform in the second stage where the number of steps is equal to or greater than the predetermined number of steps. For example, in a case where the absolute value of the difference between the representative value of the similarity in the first stage and the representative value of the similarity in the second stage does not exceed the predetermined threshold, the gait evaluation device 12 determines that the dynamic stability of gait is high. For example, in a case where the absolute value of the difference between the representative value of the similarity in the first stage and the representative value of the similarity in the second stage exceeds the predetermined threshold, the gait evaluation device 12 determines that the dynamic stability of gait is low.

For example, the gait evaluation device 12 evaluates the dynamic stability of gait according to the ratio between the representative value of the similarity between the reference target waveform and the target waveform in the first stage where the number of steps is less than the predetermined number of steps and the representative value of the similarity between the reference target waveform and the target waveform in the second stage where the number of steps is equal to or greater than the predetermined number of steps. For example, in a case where the ratio between the representative value of the similarity in the first stage and the representative value of the similarity in the second stage does not exceed the predetermined threshold, the gait evaluation device 12 determines that the dynamic stability of gait is high. For example, in a case where the ratio between the representative value of the similarity in the first stage and the representative value of the similarity in the second stage exceeds the predetermined threshold, the gait evaluation device 12 determines that the dynamic stability of gait is low.

When the time series data of the sensor data does not satisfy the reference of stable walking, the gait evaluation device 12 ends the measurement. For example, the gait evaluation device 12 is configured to detect the end of the stable walking when the value of the acceleration in the traveling direction (acceleration in the Y direction) does not exceed the first threshold for 10 seconds. The gait evaluation device 12 ends the measurement in response to the detection of the end of the stable walking.

The change in the similarity of the target waveform is reset at the end of the walking session. For example, when the user stops or changes the posture between different walking sessions, a change tendency of the similarity of the target waveform changes due to a change in the walking condition. For example, when the user exercises between different walking sessions, a change tendency of the similarity of the target waveforms changes according to the fatigue level of the user. For example, when the user takes a break between different walking sessions, a change tendency of the similarity of the target waveforms changes as the physical power of the user recovers. The degree of recovery of the physical power of the user is affected by attributes such as age and gender. Therefore, the gait evaluation device 12 verifies a change in similarity of the target waveform in a single walking session.

The gait evaluation device 12 outputs information regarding the dynamic stability of gait. For example, the gait evaluation device 12 outputs information regarding the dynamic stability of gait to a display device (not illustrated) or a mobile terminal (not illustrated). The information output to the display device is displayed on a screen of the display device or the mobile terminal. For example, the gait evaluation device 12 outputs information regarding the dynamic stability of gait to an external system (not illustrated). The information output from the gait evaluation device 12 can be used for any purpose. The communication function of outputting the information by the gait evaluation device 12 is not particularly limited.

For example, the gait evaluation device 12 is mounted on a server (not illustrated) or the like. For example, the gait evaluation device 12 may be achieved by an application server. For example, the gait evaluation device 12 may be achieved by application software or the like installed in a mobile terminal (not illustrated).

### [Measurement Device]

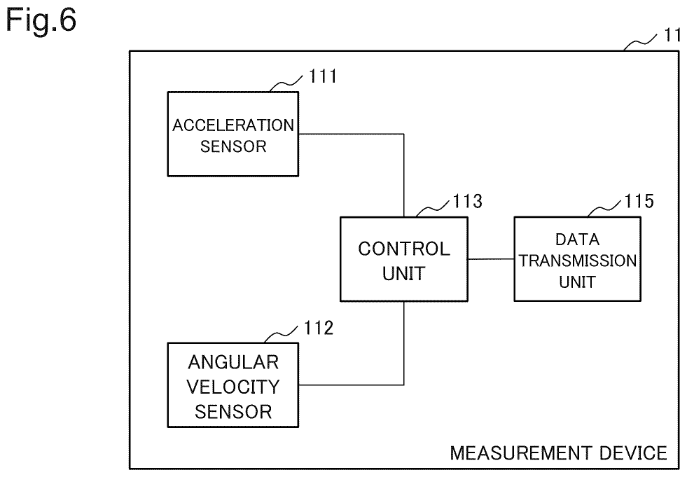

Next, the detailed configuration of the measurement device 11 will be described with reference to the drawings. FIG. 6 is a block diagram illustrating an example of the detailed configuration of the measurement device 11. The measurement device 11 includes an acceleration sensor 111, an angular velocity sensor 112, a control unit 113, and a data transmission unit 115. The measurement device 11 includes a power supply (not illustrated).

The acceleration sensor 111 is a sensor that measures accelerations (also referred to as spatial accelerations) in three axial directions. The acceleration sensor 111 outputs the measured acceleration to the control unit 113. For example, a sensor of a piezoelectric type, a piezoresistive type, a capacitance type, or the like can be used as the acceleration sensor 111. The sensor used for the acceleration sensor 111 is not limited to the measurement method as long as the sensor can measure acceleration.

The angular velocity sensor 112 is a sensor that measures an angular velocity (also referred to as a spatial angular velocity) in the three axial directions. The angular velocity sensor 112 outputs the measured angular velocity to the control unit 113. For example, a sensor of a vibration type, a capacitance type, or the like can be used as the angular velocity sensor 112. The sensor used for the angular velocity sensor 112 is not limited to the measurement method as long as the sensor can measure the angular velocity.

The control unit 113 acquires the acceleration in the three axial directions and the angular velocity around three axes from each of the acceleration sensor 111 and the angular velocity sensor 112. The control unit 113 converts the acquired acceleration and angular velocity into digital data, and outputs the converted digital data (also referred to as sensor data) to the data transmission unit 115. The sensor data includes at least acceleration data converted into digital data and angular velocity data converted into digital data. The acceleration data includes acceleration vectors in three axial directions. The angular velocity data includes angular velocity vectors around three axes. The acceleration data and the angular velocity data are associated with acquisition times of the data. The control unit 113 may be configured to output sensor data obtained by adding correction such as a mounting error, temperature correction, and linearity correction to the acquired acceleration data and angular velocity data. The control unit 113 may generate angle data around three axes using the acquired acceleration data and angular velocity data.

For example, the control unit 113 is a microcomputer or a microcontroller that controls the overall measurement device 11 or processes data. For example, the control unit 113 includes a central processing unit (CPU), a random access memory (RAM), a read only memory (ROM), a flash memory, and the like. The control unit 113 controls the acceleration sensor 111 and the angular velocity sensor 112 to measure the angular velocity and the acceleration. For example, the control unit 113 performs analog-to-digital conversion (AD conversion) on physical quantities (analog data) such as the measured angular velocity and acceleration, and stores the converted digital data in the flash memory. The physical quantity (analog data) measured by the acceleration sensor 111 and the angular velocity sensor 112 may be converted into digital data in each of the acceleration sensor 111 and the angular velocity sensor 112. The digital data stored in the flash memory is output to the data transmission unit 115 at a predetermined timing.

The data transmission unit 115 acquires sensor data from the control unit 113. The data transmission unit 115 transmits the acquired sensor data to the gait evaluation device 12. The data transmission unit 115 may transmit the sensor data to the gait evaluation device 12 via a wire such as a cable, or may transmit the sensor data to the gait evaluation device 12 via wireless communication. For example, the data transmission unit 115 is configured to transmit the sensor data to the gait evaluation device 12 via a wireless communication function (not illustrated) conforming to a standard such as Bluetooth (registered trademark) or WiFi (registered trademark). The communication function of the data transmission unit 115 may conform to a standard other than Bluetooth (registered trademark) or WiFi (registered trademark).

### [Gait Evaluation Device]

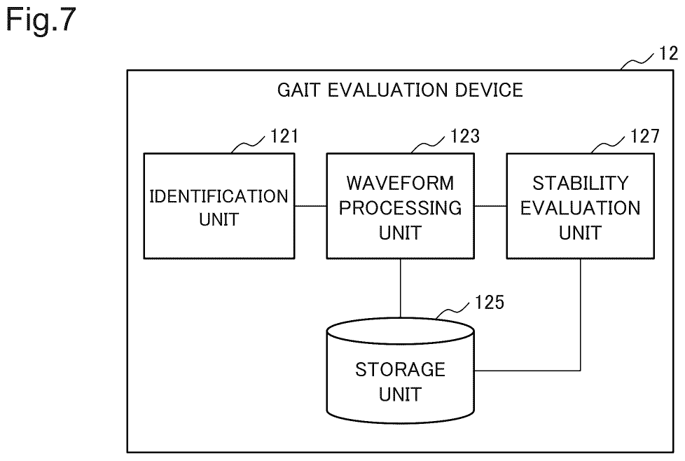

Next, the detailed configuration of the gait evaluation device 12 will be described with reference to the drawings. FIG. 7 is a block diagram illustrating an example of a configuration of the gait evaluation device 12. The gait evaluation device 12 includes an identification unit 121, a waveform processing unit 123, a storage unit 125, and a stability evaluation unit 127. In practice, a communication interface such as a reception unit that receives sensor data from the measurement device 11 and an output unit that outputs an evaluation result by the stability evaluation unit 127 is provided. In the configuration of FIG. 7, the communication interface is omitted.

The identification unit 121 acquires sensor data measured by the measurement device 11. The identification unit 121 detects the start of stable walking based on the received sensor data. For example, the identification unit 121 detects the start of stable walking according to the relationship between the peak value of the acceleration in the traveling direction (acceleration in the Y direction) and the threshold (first threshold). For example, the identification unit 121 is configured to detect the start of stable walking when the peak value of the acceleration in the traveling direction (acceleration in the Y direction) exceeds the first threshold three times.

When the time series data of the sensor data no longer satisfies the reference for stable walking, the identification unit 121 ends the measurement. For example, when the value of the acceleration in the traveling direction (acceleration in the Y direction) does not exceed the threshold for 10 seconds, the identification unit 121 detects the end of the stable walking. The identification unit 121 ends the measurement in response to the detection of the end of the stable walking.

In response to the detection of the start of stable walking by the identification unit 121, the waveform processing unit 123 generates time series data of sensor data measured by the measurement device 11 for the same walking session. The waveform processing unit 123 measures the number of steps of the user in accordance with the generation of the time series data of the sensor data. For example, the waveform processing unit 123 cuts out a waveform for one gait cycle from the time series data of the sensor data. For example, the waveform processing unit 123 cuts out a waveform for one step from the time series data of the sensor data. For example, the waveform processing unit 123 cuts out a waveform for one stride from the time series data of the sensor data. The waveform processing unit 123 normalizes the horizontal axis (time) of the cut-out waveform to a gait cycle of 0 to 100%. The waveform processing unit 123 normalizes the vertical axis (intensity) of the cut-out waveform with the maximum intensity as a reference.

The waveform processing unit 123 extracts a waveform (also referred to as a target waveform) included in the evaluation target segment of the dynamic stability of gait from the normalized waveform (also referred to as a gait waveform) for one gait cycle. For example, the waveform processing unit 123 extracts a waveform during the swing phase as the target waveform. In the period of the swing phase, since the measurement device 11 floats in the air, there are many variations in acceleration, and a difference is likely to occur for each number of steps. The change in the dynamic stability of gait is likely to appear in a segment where the difference for each number of steps is likely to occur. Therefore, the waveform of the period of the swing phase is suitable for verifying the transition of similarity.

For example, the waveform processing unit 123 may extract the target waveform relevant to the gait period included in the evaluation target segment based on the gait event detected from the time series data of the sensor data. For example, the waveform processing unit 123 detects toe off, foot adjacent, tibia vertical, and heel strike from the gait waveform. For example, the waveform processing unit 123 specifies a segment between the toe off and the foot adjacent as the initial swing period. For example, the waveform processing unit 123 specifies a segment between the foot adjacent and the tibia vertical as the mid-swing period. For example, the waveform processing unit 123 specifies a segment between the tibia vertical and the heel strike as the end terminal swing period.

The change in the dynamic stability of gait tends to depend on the fatigue level of muscles involved in walking. For example, the waveform processing unit 123 may extract the target waveform included in the evaluation target segment (gait period) set according to the type of the muscle of the determination target of the fatigue level. For example, in a case where the muscle of which the fatigue level is to be determined is the abduction muscle, the mid-swing period in which the feature of foot distribution appears may be set as the evaluation target segment. In this case, the waveform processing unit 123 extracts the target waveform included in the mid-swing period as the evaluation target segment. For example, in a case where the muscle of the determination target of the fatigue level is the iliopsoas muscle, an initial swing period in which the feature of a motion of swinging roughly forward appears may be set as the evaluation target segment. In this case, the waveform processing unit 123 extracts the target waveform included in the initial swing period that is the evaluation target segment.

The waveform processing unit 123 extracts the target waveform in all the gait cycles in the segment (also referred to as a walking session) from the detection of the start of the stable walking to the detection of the end of the stable walking. The waveform processing unit 123 stores the extracted target waveform in the storage unit 125. The waveform processing unit 123 may be configured to output the extracted target waveform to the stability evaluation unit 127. The waveform processing unit 123 may be configured to transmit the extracted target waveform to an external server (not illustrated) or database (not illustrated).

The storage unit 125 stores the target waveform extracted by the waveform processing unit 123. The target waveform stored in the storage unit 125 is used for similarity evaluation by the stability evaluation unit 127. The storage unit 125 may be omitted in a case where the target waveform is configured to be output from the waveform processing unit 123 to the stability evaluation unit 127 or in a case where the target waveform is transmitted from the waveform processing unit 123 to an external server or database.

The stability evaluation unit 127 acquires a target waveform used for evaluation of similarity from the storage unit 125. The waveform processing unit 123 evaluates the dynamic stability of gait based on a change in similarity of the target waveform in the walking session. The stability evaluation unit 127 may be configured to acquire the target waveform from the waveform processing unit 123.

The stability evaluation unit 127 calculates similarity between a reference target waveform extracted from a gait waveform of a reference gait cycle and a target waveform extracted from a gait waveform of a series of other gait cycles in the same walking session. For example, the stability evaluation unit 127 calculates similarity between a reference target waveform extracted from a gait waveform in the first gait cycle and a target waveform extracted from a gait waveform in a series of subsequent gait cycles in the same walking session. For example, the stability evaluation unit 127 may calculate similarity between a reference target waveform extracted from a gait waveform after several gait cycles and a target waveform extracted from a gait waveform in a series of subsequent gait cycles in the same walking session. For example, the stability evaluation unit 127 may calculate similarity between a representative value of a target waveform extracted from a gait waveform for several gait cycles and a representative value of a target waveform extracted from a gait waveform of a series of subsequent gait cycles in the same walking session. The stability evaluation unit 127 may calculate similarity for each stride or the number of steps.

For example, the stability evaluation unit 127 calculates, as similarity, Pearson's linear correlation coefficient between a reference target waveform extracted from a gait waveform of a reference gait cycle and a target waveform extracted from a gait waveform of a series of other gait cycles in the same session. For example, the stability evaluation unit 127 may use the target waveform as a vector and calculate the similarity according to the angle between the vectors. For example, when two different target waveforms have a similarity relationship, the angle between the vectors of the target waveforms is 0 degrees. For example, as the similarity between two different target waveforms decreases, the angle between the vectors of the target waveforms increases. For example, the stability evaluation unit 127 may set the target waveform as one data group and calculate the intra-class correlation coefficients of the two target waveforms as similarity. For example, in a case where the intra-class correlation coefficients of the two target waveforms completely matches, the stability evaluation unit 127 determines that the target waveforms match. For example, in a case where the intra-class correlation coefficients of the two target waveforms do not completely match with each other, the stability evaluation unit 127 determines that the target waveforms do not match with each other.

For example, the stability evaluation unit 127 compares the waveforms in the segment to be compared based on values of acceleration, angular velocity, velocity, position, angle, and the like of the target waveform. For example, the stability evaluation unit 127 may select the strength to be compared according to the region or movement of the muscle to be evaluated. For example, the stability evaluation unit 127 compares the waveform of the segment to be compared based on the intensities of the acceleration in the lateral direction (acceleration in the X direction), the acceleration in the traveling direction (acceleration in the Y direction), and the acceleration in the vertical direction (acceleration in the Z direction). The dynamic stability of gait is characterized by the acceleration in the lateral direction (acceleration in the X direction). Therefore, the stability evaluation unit 127 may compare the waveforms in the segment to be compared based on the intensity of the acceleration in the lateral direction (acceleration in the X direction). For example, the stability evaluation unit 127 may increase the weight of the acceleration in the lateral direction (acceleration in the X direction) among the intensities of the acceleration in the lateral direction (acceleration in the X direction), the acceleration in the traveling direction (acceleration in the Y direction), and the acceleration in the vertical direction (acceleration in the Z direction), and compare the waveforms in the segment to be compared.

The stability evaluation unit 127 evaluates the dynamic stability of gait of the user in response to the transition of the similarity of the target waveform accompanying the walking of the user. For example, the stability evaluation unit 127 evaluates the dynamic stability of gait according to a decreasing tendency of the similarity of the target waveform. For example, in a case where the decreasing rate of the similarity of the target waveform falls below a predetermined threshold, the stability evaluation unit 127 determines that the dynamic stability of gait has decreased. For example, in a case where the decreasing rate of the similarity of the target waveform falls below a predetermined threshold for a predetermined period, the stability evaluation unit 127 determines that the dynamic stability of gait has decreased.

For example, the stability evaluation unit 127 evaluates the dynamic stability of gait according to a change in similarity with the progress of walking. For example, the stability evaluation unit 127 evaluates the dynamic stability of gait according to the difference between the representative value of the similarity between the reference target waveform and the target waveform in the first stage where the number of steps is less than the predetermined number of steps and the representative value of the similarity between the reference target waveform and the target waveform in the second stage where the number of steps is equal to or greater than the predetermined number of steps. For example, the stability evaluation unit 127 evaluates the dynamic stability of gait by comparing representative values such as an average value, a mode value, and a median value. For example, the stability evaluation unit 127 evaluates the dynamic stability of gait by comparing average values such as an arithmetic mean, a geometric mean, a harmonic mean, and a logarithmic mean.

For example, the stability evaluation unit 127 evaluates the dynamic stability of gait according to the difference between the representative value of the similarity between the reference target waveform and the target waveform in the first stage where the number of steps is less than the predetermined number of steps and the representative value of the similarity between the reference target waveform and the target waveform in the second stage where the number of steps is equal to or greater than the predetermined number of steps. For example, in a case where the absolute value of the difference between the representative value of the similarity in the first stage and the representative value of the similarity in the second stage does not exceed the predetermined threshold, the stability evaluation unit 127 determines that the dynamic stability of gait is high. For example, in a case where the absolute value of the difference between the representative value of the similarity in the first stage and the representative value of the similarity in the second stage exceeds the predetermined threshold, the stability evaluation unit 127 determines that the dynamic stability of gait is low.

For example, the stability evaluation unit 127 evaluates the dynamic stability of gait according to the ratio between the representative value of the similarity between the reference target waveform and the target waveform in the first stage where the number of steps is less than the predetermined number of steps and the representative value of the similarity between the reference target waveform and the target waveform in the second stage where the number of steps is equal to or greater than the predetermined number of steps. For example, in a case where the ratio between the representative value of the similarity in the first stage and the representative value of the similarity in the second stage does not exceed the predetermined threshold, the stability evaluation unit 127 determines that the dynamic stability of gait is high. For example, in a case where the ratio between the representative value of the similarity in the first stage and the representative value of the similarity in the second stage exceeds the predetermined threshold, the stability evaluation unit 127 determines that the dynamic stability of gait is low.

The stability evaluation unit 127 outputs information regarding the dynamic stability of gait. For example, the information regarding the dynamic stability of gait is output to a display device (not illustrated) or a mobile terminal (not illustrated). The information output to the display device is displayed on a screen of the display device or the mobile terminal. For example, the information regarding the dynamic stability of gait is output to an external system (not shown). The information regarding the dynamic stability of gait can be used for any application. The communication function for outputting the information regarding the dynamic stability of gait is not particularly limited.

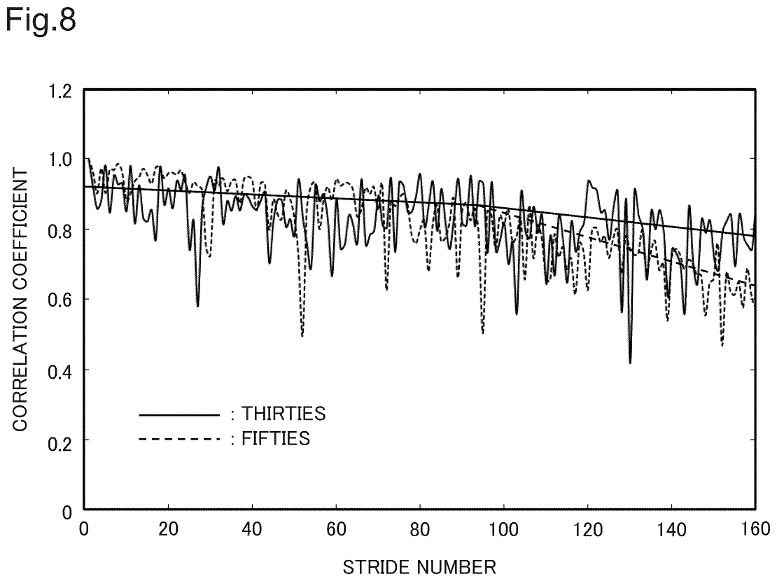

FIG. 8 is a graph illustrating a difference in change of the target waveform according to the age of a pedestrian. FIG. 8 shows the transition of the target waveform verified for subjects in their thirties and fifties. FIG. 8 relates to a transition of similarity of a target waveform in a segment from the terminal stance period to the terminal swing period among time series data of sensor data measured by walking 200 meters (m) in a time zone of good physical condition in the morning. In the example of FIG. 8, the target waveform extracted from the gait waveform in the first gait cycle is the reference target waveform. The horizontal axis of the graph of FIG. 8 is the stride number. The vertical axis of the graph of FIG. 8 is a correlation coefficient between the target waveform extracted from the gait waveform of the first step and the target waveform extracted for each number of steps. In the segment from the terminal stance period to the terminal swing period, since the measurement device 11 floats in the air, there are many variations in acceleration, and a difference is likely to occur for each number of steps. Therefore, the segment from the terminal stance period to the terminal swing period is suitable for evaluating a long-term change.

In FIG. 8, the baseline of the similarity of the target waveform in the walking of the subject in his/her thirties is indicated by a solid line, and the baseline of the similarity of the target waveform in the walking of the subject in his/her fifties is indicated by a broken line. As illustrated in FIG. 8, regardless of the age, the decreasing rate of the similarity of the target waveform (the slope of the baseline) tends to increase as the stride number increases. In the example of FIG. 8, there is no significant difference in the transition of the similarity between the target waveforms of the thirties and the fifties up to about before 100 steps. However, the subject in his or her fifties tends to have a decrease in the similarity of the target waveform as compared with the subject in his or her thirties around the time when the steps exceed 100. The decrease in similarity of the target waveform is relevant to a decrease in dynamic stability of gait. That is, the difference in the dynamic stability of gait according to age tends to appear in the second half of the walking session.

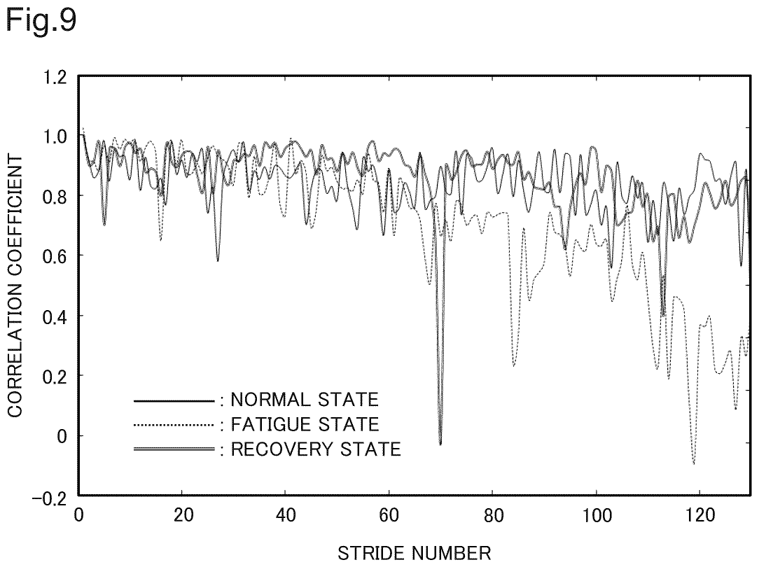

FIG. 9 is a graph illustrating a difference in change of a target waveform due to a difference in physical condition of a pedestrian. FIG. 9 shows the transition of the target waveform verified for subjects in their thirties. FIG. 9 relates to a transition of similarity of a target waveform in a segment from the terminal stance period to the terminal swing period among time series data of sensor data measured by walking 200 meters (m). In the example of FIG. 9, the target waveform extracted from the gait waveform in the first gait cycle is the reference target waveform. The horizontal axis of the graph of FIG. 9 is the stride number. The vertical axis of the graph of FIG. 9 is a correlation coefficient between the target waveform extracted from the gait waveform of the first step and the target waveform extracted for each number of steps.

FIG. 9 illustrates a difference in the transition of the target waveform in a time zone when the physical condition is good in the morning (normal state), immediately after the abduction muscles are fatigued in the muscle strength training (fatigue state), and after taking a break for 4 hours after the muscle strength training (recovery). In FIG. 9, the difference in the transition of the target waveform at the normal state is indicated by a solid line, the difference in the transition of the target waveform in the fatigue state is indicated by a dotted line, and the difference in the transition of the target waveform at the time of recovery is indicated by a double line. As illustrated in FIG. 9, the similarity of the target waveform tends to decrease as the stride number increases regardless of the physical condition. In the example of FIG. 9, no significant difference is observed in the transition of the similarity of the target waveform up to about 60 steps. However, from around the time when the steps exceed 70, a decrease in the correlation coefficient in the fatigue state is conspicuous as compared with others. Then, from around the time when the steps exceed 100, the decrease in the correlation coefficient in the fatigue state becomes more remarkable. The decrease in similarity of the target waveform is relevant to a decrease in dynamic stability of gait. That is, the difference in the dynamic stability of gait according to the physical condition tends to appear in the second half of the walking session.

As in the example of FIGS. 8 and 9, the stability evaluation unit 127 evaluates the dynamic stability of gait based on the long-term similarity of the target waveform within one walking session instead of the similarity of the target waveform in a short period. Therefore, the stability evaluation unit 127 can evaluate the long-term dynamic stability of gait that cannot be verified by a short-term change.

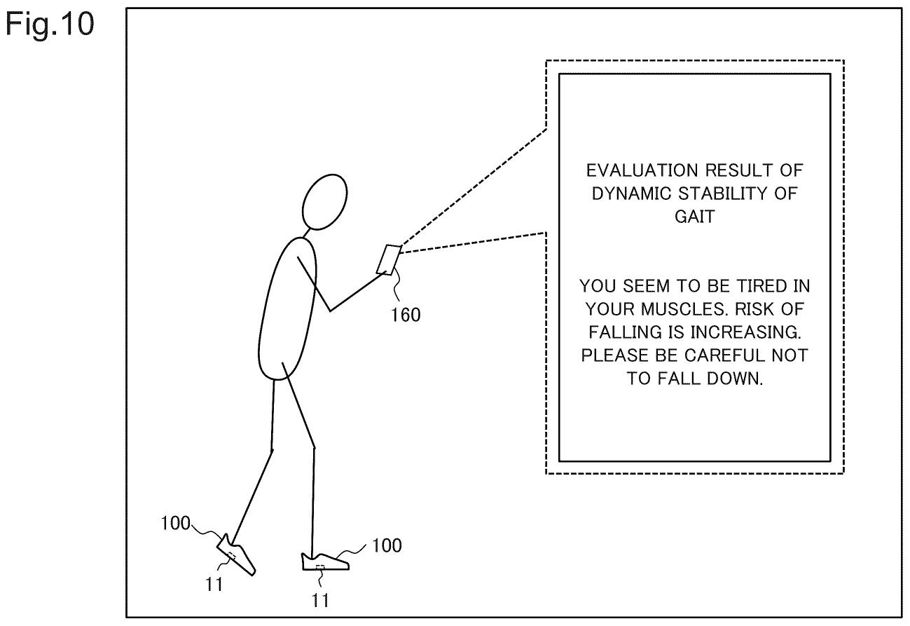

FIG. 10 is a conceptual diagram illustrating an example of a usage scene of the gait measurement system 1. FIG. 10 is an example in which information regarding the evaluation result of the dynamic stability of gait of the user is displayed on the screen of a mobile terminal 160 of the user wearing the shoe 100 on which the measurement device 11 is installed. In the example of FIG. 10, in accordance with the evaluation result of the gait measurement system 1, the information “You seem to be tired in your muscles. The risk of falling is increasing. Please be careful not to fall down.” is displayed in the screen of the mobile terminal 160 as the evaluation result of the dynamic stability of gait. The user who has confirmed the information displayed on the screen of the mobile terminal 160 can take an action according to the information. For example, the pedestrian who has confirmed the information displayed on the screen of the mobile terminal 160 can continue walking while paying attention to falling or take a break while avoiding the risk of falling according to the content of the information.

### (Operation)

Next, an operation of the gait measurement system 1 will be described with reference to the drawings. Description of operations of the measurement device 11 will be omitted. Hereinafter, an example will be described in which the dynamic stability of gait is evaluated each time the sensor data measured by the measurement device 11 is acquired. The following operation of the gait measurement system 1 may include an operation different from the above description of the configuration.

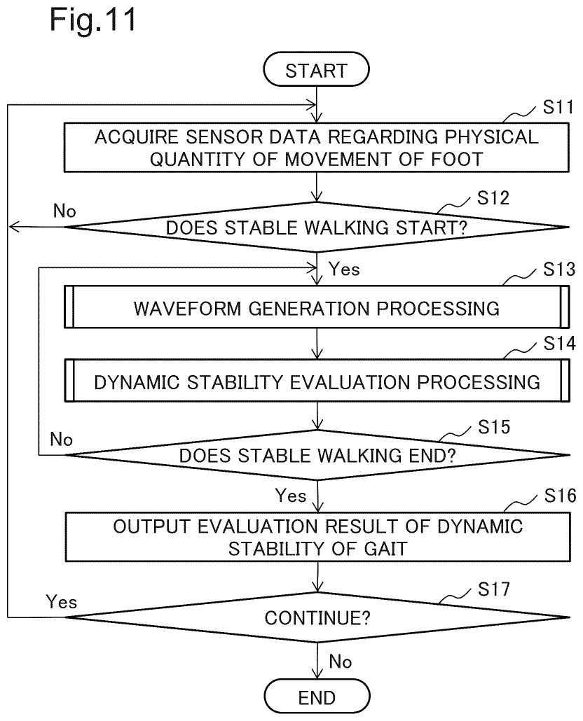

FIG. 11 is a flowchart for explaining an example of the operation of the gait evaluation device 12. In the description along the flowchart of FIG. 11, the identification unit 121, the waveform processing unit 123, and the stability evaluation unit 127 included in the gait evaluation device 12 will be described as operation subjects.

In FIG. 11, the identification unit 121 acquires sensor data regarding the physical quantity of the movement of the foot (step S11).

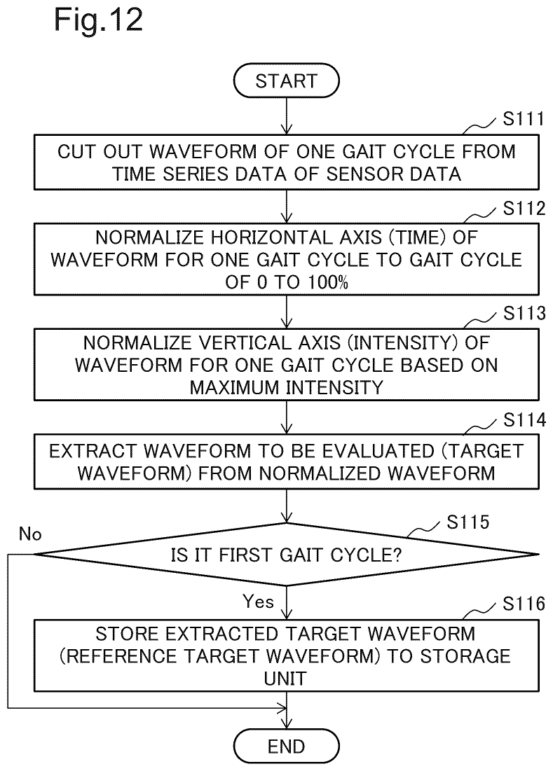

When the start of the walking session (start of stable walking) is detected by the identification unit 121 (Yes in step S12), the waveform processing unit 123 executes waveform generation processing (step S13). The waveform generation processing in step S13 will be described later (FIG. 12). When the start of the walking session (start of stable walking) is not detected (No in step S12), the process returns to step S11.

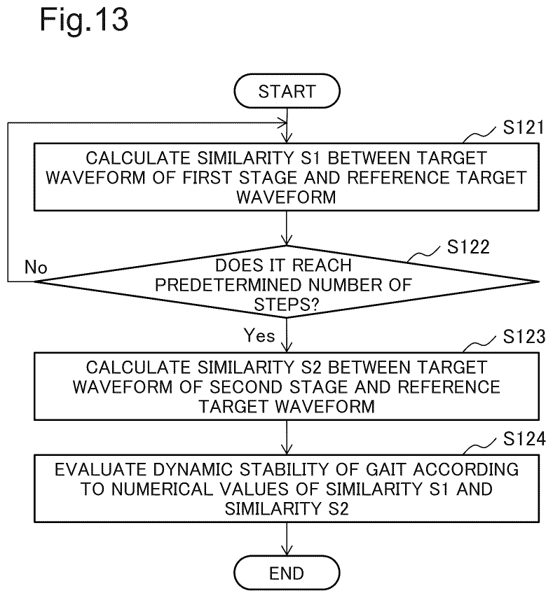

After step S13, the stability evaluation unit 127 executes dynamic stability evaluation processing (step S14). Details of the dynamic stability evaluation processing in step S14 will be described later (FIG. 13).

When the end of the walking session (end of stable walking) is detected by the identification unit 121 (Yes in step S15), the stability evaluation unit 127 outputs information regarding the evaluation result of the dynamic stability of gait. In a case where the end of the walking session (end of stable walking) is not detected by the identification unit 121 (No in step S15), the process returns to step S14.

After step S16, when the process is continued (Yes in step S17), the process returns to step S11. When the process is not continued (No in step S17), the process according to the flowchart of FIG. 11 is ended. Whether to continue the process may be determined based on a preset criterion.

### [Waveform Generation Processing]

FIG. 12 is a flowchart for explaining the waveform generation processing (step S13 in FIG. 11). In the processing description along the flowchart of FIG. 12, the waveform processing unit 123 included in the gait evaluation device 12 will be described as an operation subject.

In FIG. 12, first, the waveform processing unit 123 cuts out a waveform for one gait cycle from the time series data of the sensor data (step S111).

Next, the waveform processing unit 123 normalizes the time (horizontal axis) of the waveform for one gait cycle to a gait cycle of 0 to 100% (step S112).

Next, the waveform processing unit 123 normalizes the intensity (vertical axis) of the waveform for one gait cycle based on the maximum intensity (step S113). For example, the waveform processing unit 123 normalizes the intensity of the waveform for one gait cycle with the maximum intensity as 1.

Next, the waveform processing unit 123 extracts a waveform to be evaluated for the dynamic stability of gait (also referred to as a target waveform) from the normalized waveform (step S114).

In the case of the first gait cycle (Yes in step S115), the waveform processing unit 123 stores the extracted target waveform (reference target waveform) in the storage unit 125 (step S116). In step S116, the process according to the flowchart in FIG. 12 ends (the process proceeds to step S14 in FIG. 11). In a case where it is not the first gait cycle (No in step S115), the process along the flowchart of FIG. 12 is ended.

### [Dynamic Stability Evaluation Processing]

FIG. 13 is a flowchart for explaining the dynamic stability evaluation processing (step S14 in FIG. 11). FIG. 13 illustrates an example of evaluating the dynamic stability of gait by calculating similarity in stages before and after a predetermined number of steps and comparing the similarities calculated for each stage. In the description of the processing along the flowchart of FIG. 13, the stability evaluation unit 127 included in the gait evaluation device 12 will be described as an operation subject.

In FIG. 13, first, the stability evaluation unit 127 calculates similarity S1 between the target waveform of the first stage and the reference target waveform (step S121). The first stage is a stage of a segment from detection of stable walking to a predetermined number of steps.

When the predetermined number of steps has been reached (Yes in step S122), the stability evaluation unit 127 calculates similarity S2 between the second-stage target waveform and the reference target waveform (step S123). When the predetermined number of steps has not been reached (No in step S122), the process returns to step S121.

After step S123, the stability evaluation unit 127 evaluates the dynamic stability of gait according to the numerical values of the similarity S1 and the similarity S2 (step S124). For example, the stability evaluation unit 127 evaluates the dynamic stability of gait based on representative values of the similarity S1 and the similarity S2. In step S124, the process according to the flowchart in FIG. 13 ends (the process proceeds to step S15 in FIG. 11).

In the description of FIGS. 11 to 13, an example has been described in which the dynamic stability of gait is evaluated each time the sensor data measured by the measurement device 11 is acquired. In a case where the dynamic stability of gait is evaluated for a sensor data group in which a walking session is known in advance, the waveform generation processing and the dynamic stability evaluation processing may be performed without detecting/ending stable walking.

As described above, the gait measurement system of the present example embodiment includes the measurement device and the gait evaluation device. The measurement device is disposed on the user's footwear. The measurement device measures the spatial acceleration and the spatial angular velocity according to the gait of the user. The measurement device generates sensor data based on the measured spatial acceleration and spatial angular velocity. The measurement device outputs the generated sensor data to the gait evaluation device. The gait evaluation device includes an identification unit, a waveform processing unit, and a stability evaluation unit. The identification unit identifies a walking session in which stable walking is performed based on the sensor data regarding the movement of the foot. The waveform processing unit extracts a target waveform included in an evaluation target segment of the dynamic stability of gait from time series data of sensor data measured for the same walking session for each gait cycle. The stability evaluation unit evaluates the dynamic stability of gait in response to the transition of the similarity of the target waveform extracted for each gait cycle. The stability evaluation unit outputs an evaluation result of the dynamic stability of gait.

In the present example embodiment, the dynamic stability of gait is evaluated in response to the transition of the similarity of the target waveform extracted from the evaluation target segment for each gait cycle included in the same walking session. In the present example embodiment, the dynamic stability of gait is evaluated in response to the transition of the similarity of the target waveform, instead of individually comparing the target waveform for each gait cycle. Therefore, according to the present example embodiment, the dynamic stability of gait can be accurately evaluated without being affected by an accidental gait variation. According to the present example embodiment, since the evaluation is performed for each walking session, the dynamic stability of gait can be appropriately evaluated according to the change in physical condition between the walking sessions.

In one aspect of the present example embodiment, the waveform processing unit generates a gait waveform in which the gait cycle and the intensity are normalized using the time series data of the sensor data. The waveform processing unit extracts a target waveform included in the evaluation target segment from the generated gait waveform. According to the present aspect, since the target waveform is normalized, the similarity of the target waveform can be verified more accurately. Therefore, according to the present aspect, the dynamic stability of gait can be more accurately evaluated.

In one aspect of the present example embodiment, the stability evaluation unit evaluates the dynamic stability of gait in response to the transition of the similarity between the reference target waveform in the initial stage of the walking session and a series of target waveforms included in the walking session. For example, the stability evaluation unit uses an intra-class correlation coefficient between the reference target waveform and a series of target waveforms as an index of similarity. According to the present aspect, it is possible to verify the transition of the similarity of a series of target waveforms with reference to a single reference target waveform. Therefore, according to the present aspect, the dynamic stability of gait can be more accurately evaluated.

In one aspect of the present example embodiment, the stability evaluation unit compares the representative value of the similarity between the reference target waveform and the target waveform in the first stage where the number of steps is less than the predetermined number of steps with the representative value of the similarity between the reference target waveform and the target waveform in the second stage where the number of steps is equal to or greater than the predetermined number of steps. The stability evaluation unit evaluates the dynamic stability of gait according to the comparison result between the representative value of the similarity in the first stage and the representative value of the similarity in the second stage. According to the present aspect, by evaluating the dynamic stability of gait according to the comparison result of the representative values of the similarities of the target waveform in the first stage and the second stage, it is possible to clearly verify the change in the similarity before and after the predetermined number of steps.

In one aspect of the present example embodiment, the waveform processing unit extracts the target waveform from the evaluation target segment set according to the type of the muscle of the determination target of the fatigue level. The stability evaluation unit determines the fatigue level of the muscle to be determined in response to a long-term transition of the similarity of the target waveform. The stability evaluation unit outputs a determination result of the fatigue level of the muscle to be determined. According to the present aspect, the fatigue level of the muscle to be determined can be appropriately determined by extracting a target waveform suitable for determination according to the type of the muscle to be determined for the fatigue level.

### Second Example Embodiment

Next, a gait measurement system 2 according to a second example embodiment will be described with reference to the drawings. The gait measurement system 2 of the present example embodiment is different from that of the first example embodiment in that the dynamic stability of gait is evaluated using a similarity matrix in which similarity between pairs of different target waveforms is mapped, instead of using a single reference target waveform as a reference.

### (Configuration)

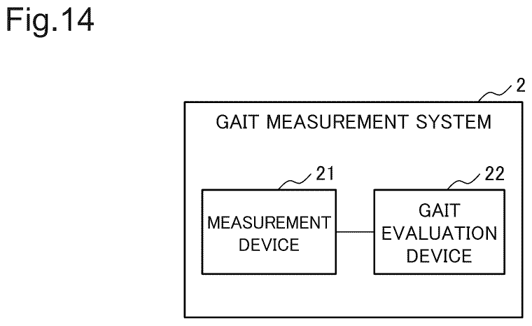

FIG. 14 is a block diagram illustrating a configuration of the gait measurement system 2 according to the present example embodiment. The gait measurement system 2 includes a measurement device 21 and a gait evaluation device 22. The gait evaluation device 22 may be connected to the measurement device 21 in a wired manner or in a wireless manner. The measurement device 21 and the gait evaluation device 22 may be configured by a single device. The gait measurement system 2 may include only the gait evaluation device 22 except for the measurement device 21.

The measurement device 21 has a configuration similar to that of the measurement device 11 according to the first example embodiment. The measurement device 21 is installed on the foot portion. The measurement device 21 measures acceleration measured by the acceleration sensor (also referred to as spatial acceleration) and an angular velocity measured by the angular velocity sensor (also referred to as spatial angular velocity) as physical quantities regarding the movement of the foot of the user wearing the footwear. The physical quantity regarding the movement of the foot measured by the measurement device 21 includes a velocity, an angle, and a position (trajectory) calculated by integrating the acceleration and the angular velocity. The measurement device 21 converts the measured physical quantity into digital data (also referred to as sensor data). The measurement device 21 transmits the converted sensor data to the gait evaluation device 22.

The gait evaluation device 22 receives sensor data from the measurement device 21. The gait evaluation device 22 detects the start of stable walking based on the received sensor data. For example, the gait evaluation device 22 detects the start of stable walking according to a relationship between a peak value of the acceleration in the traveling direction (acceleration in the Y direction) and a threshold (also referred to as a first threshold). For example, the gait evaluation device 22 is configured to detect the start of stable walking when the peak value of the acceleration in the traveling direction (acceleration in the Y direction) exceeds the first threshold three times. The gait evaluation device 22 evaluates the dynamic stability of gait in a segment (also referred to as a walking session) from a time point at which the start of stable walking is detected to a time point at which the end of stable walking is detected.

When detecting the start of stable walking, the gait evaluation device 22 generates time series data of sensor data measured by the measurement device 21. The gait evaluation device 22 measures the number of steps of the user in accordance with the generation of the time series data of the sensor data. The gait evaluation device 22 cuts out a waveform for one gait cycle from time series data of sensor data for one gait cycle. The gait evaluation device 22 normalizes the horizontal axis (time) of the waveform for one gait cycle to a gait cycle of 0 to 100%. The gait evaluation device 22 normalizes the vertical axis (intensity) of the waveform for one gait cycle with the maximum intensity as a reference.

The gait evaluation device 22 extracts a waveform (also referred to as a target waveform) to be evaluated for the dynamic stability of gait from a normalized waveform (also referred to as a gait waveform) for one gait cycle. For example, the gait evaluation device 22 extracts a waveform of a period of the swing phase as the target waveform. The gait evaluation device 22 extracts a target waveform in a plurality of gait cycles in one walking session. The gait evaluation device 22 evaluates dynamic stability of gait based on a change in similarity of a target waveform in a walking session. For example, the gait evaluation device 22 tracks the similarity of the short-term, mid-term, and long-term target waveforms in the same walking session.

The gait evaluation device 22 calculates similarity in a brute-force manner for a plurality of target waveforms extracted in the same walking session. The gait evaluation device 22 generates a matrix of similarity (also referred to as a similarity matrix) regarding a plurality of pairs of target waveforms for which similarity is calculated. Details of the similarity matrix will be described later. The gait evaluation device 22 evaluates the dynamic stability of gait based on the features appearing in the similarity matrix.

When the time series data of the sensor data does not satisfy the reference of stable walking, the gait evaluation device 22 ends the measurement. For example, the gait evaluation device 22 detects the end of the stable walking when the value of the acceleration in the traveling direction (acceleration in the Y direction) does not exceed the first threshold for 10 seconds. The gait evaluation device 22 ends the measurement in response to the detection of the end of the stable walking.

The gait evaluation device 22 outputs information regarding the dynamic stability of gait. For example, the gait evaluation device 22 outputs information regarding the dynamic stability of gait to a display device (not illustrated) or a mobile terminal (not illustrated). The information output to the display device is displayed on a screen of the display device or the mobile terminal. For example, the gait evaluation device 22 outputs information regarding the dynamic stability of gait to an external system (not illustrated). The information output from the gait evaluation device 22 can be used for any purpose. The communication function of outputting the information by the gait evaluation device 22 is not particularly limited.

For example, the gait evaluation device 22 is mounted on a server (not illustrated) or the like. For example, the gait evaluation device 22 may be achieved by an application server. For example, the gait evaluation device 22 may be achieved by application software or the like installed in a mobile terminal (not illustrated). The measurement is ended in response to the detection of the end of the constant walking.

### [Gait Evaluation Device]

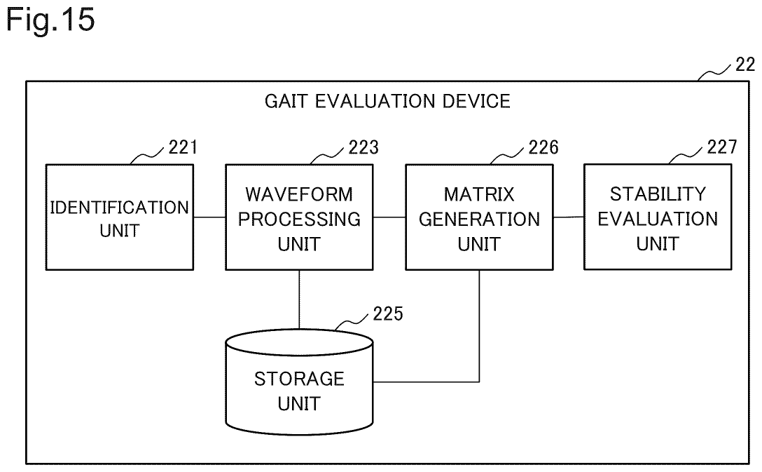

Next, the detailed configuration of the gait evaluation device 22 will be described with reference to the drawings. FIG. 15 is a block diagram illustrating an example of a configuration of the gait evaluation device 22. The gait evaluation device 22 includes an identification unit 221, a waveform processing unit 223, a storage unit 225, a matrix generation unit 226, and a stability evaluation unit 227. In practice, a communication interface such as a reception unit that receives sensor data from the measurement device 21 and an output unit that outputs an evaluation result by the stability evaluation unit 227 is provided. In the configuration of FIG. 15, the communication interface is omitted.

The identification unit 221 has a configuration similar to that of the identification unit 121 according to the first example embodiment. The identification unit 221 acquires sensor data measured by the measurement device 21. The identification unit 221 detects the start of stable walking based on the received sensor data. When the time series data of the sensor data no longer satisfies the reference for stable walking, the identification unit 221 ends the measurement.

The waveform processing unit 223 has a configuration similar to that of the waveform processing unit 123 of the first example embodiment. The waveform processing unit 223 generates time series data of sensor data measured by the measurement device 21 in response to the detection of the feature of stable walking by the identification unit 221. The waveform processing unit 223 measures the number of steps of the user in accordance with the generation of the time series data of the sensor data. The waveform processing unit 223 cuts out a waveform for one gait cycle from time series data of sensor data for one gait cycle. The waveform processing unit 223 normalizes the horizontal axis (time) of the waveform for one gait cycle to a gait cycle of 0 to 100%. The waveform processing unit 223 normalizes the vertical axis (intensity) of the waveform for one gait cycle with the maximum intensity as a reference.

The waveform processing unit 223 extracts a waveform (also referred to as a target waveform) to be evaluated for the dynamic stability of gait from a normalized waveform (also referred to as a gait waveform) for one gait cycle. The waveform processing unit 223 extracts the target waveform for all the gait cycles. The waveform processing unit 223 stores the extracted target waveform in the storage unit 225. The waveform processing unit 223 may be configured to output the extracted target waveform to the stability evaluation unit 227. The waveform processing unit 223 may be configured to transmit the extracted target waveform to an external server (not illustrated) or an external database (not illustrated).

The storage unit 225 has a configuration similar to that of the storage unit 125 according to the first example embodiment. The storage unit 225 stores the target waveform extracted by the waveform processing unit 223. The target waveform stored in the storage unit 225 is used for similarity evaluation by the stability evaluation unit 227. The storage unit 225 may be omitted in a case where the waveform processing unit 223 is configured to output to the stability evaluation unit 227 or in a case where the waveform processing unit 223 transmits to an external server or database.

The matrix generation unit 226 acquires a target waveform used for similarity evaluation from the storage unit 225. The matrix generation unit 226 calculates similarity in a brute-force manner for a plurality of pairs of target waveforms extracted in the same walking session. The gait evaluation device 22 generates a matrix (also referred to as a similarity matrix) obtained by two-dimensionally mapping similarity regarding a plurality of pairs of target waveforms for which similarity is calculated. In the similarity matrix, the magnitude relationship of the similarity of the target waveform is represented by light and dark (shading). For example, in the similarity matrix, the higher the similarity of the target waveform, the brighter (white) the target waveform, and the lower the similarity of the target waveform, the darker (black) the target waveform. The magnitude relationship of the similarity of the target waveform may be displayed by a difference in color instead of light and dark (shading).

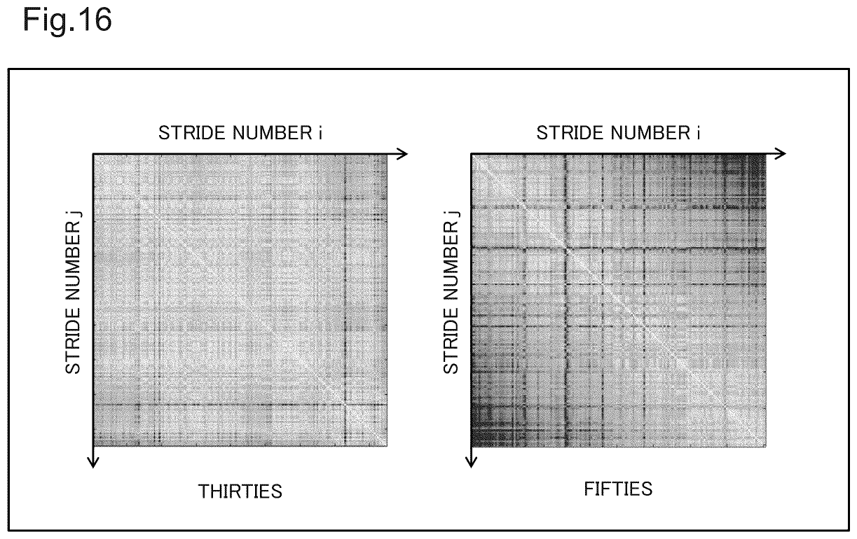

FIG. 16 is a conceptual diagram illustrating an example of a similarity matrix generated by the matrix generation unit 226. The similarity matrix of FIG. 16 is obtained by mapping the similarity between the target waveforms of all the stride numbers i and the target waveforms of all the stride numbers j extracted in the same walking session (i and j are natural numbers). FIG. 16 illustrates a difference in change in similarity of the target waveform according to the age of the pedestrian. FIG. 16 shows a similarity matrix for subjects in their thirties (left side) and a similarity matrix for subjects in their fifties (right side). FIG. 16 is a similarity matrix of the target waveform in the segment from the terminal stance period to the terminal swing period among the time series data of the sensor data measured by walking 200 meters (m) in the time zone when the physical condition is good in the morning. Since the target waveforms of the same stride number (i=j) are the same, their similarity is displayed with the maximum brightness. Therefore, a bright diagonal line appears from the upper left to the lower right of the similarity matrix. In the similarity matrix, a combination of the same stride number appears at a line-symmetric position with respect to a diagonal line. That is, the similarity matrix is line-symmetric with respect to the diagonal line from the upper left to the lower right. Hereinafter, description will be given focusing on an upper right region with the diagonal line interposed therebetween.

In the similarity matrix of FIG. 16, for both subjects in their thirties (left side) and subjects in their fifties (right side), the similarity of the target waveforms tends to decrease (darken) as the stride number increases. Compared with subjects in their thirties (left side), subjects in their fifties (right side) have a larger area of dark regions in the upper right of the similarity matrix. This indicates that the subjects in their fifties start to decrease the similarity of the target waveform earlier than the subjects in their thirties. The decrease in similarity of the target waveform tends to depend on muscle strength such as abduction muscles. It is presumed that a difference in muscle strength due to a difference in age is reflected in the similarity matrix of FIG. 16.

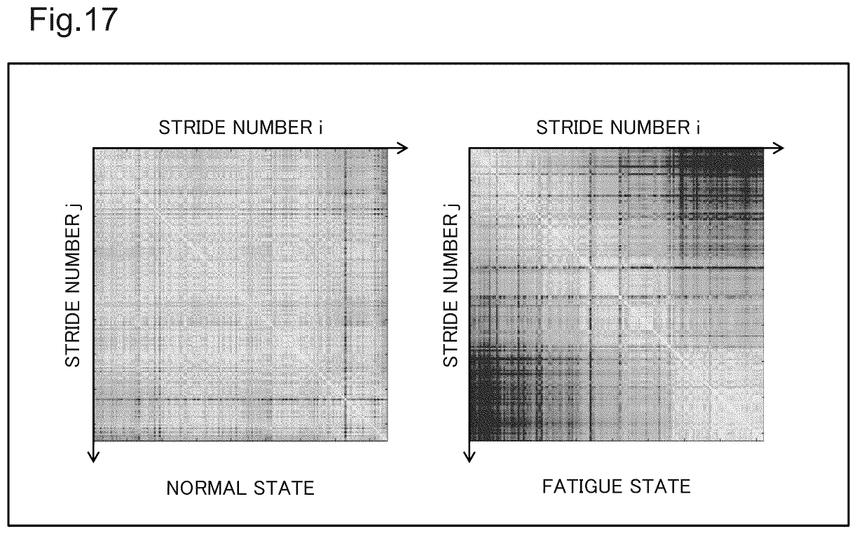

FIG. 17 is a conceptual diagram illustrating another example of the similarity matrix generated by the matrix generation unit 226. FIG. 17 shows a similarity matrix for subjects in their thirties. FIG. 17 illustrates a difference in similarity between target waveforms due to a difference in physical condition of a pedestrian. FIG. 17 illustrates a similarity matrix (left side) based on sensor data measured in a good physical condition time zone (normal state) in the morning, and a similarity matrix (right side) based on sensor data measured immediately after the abduction muscle is fatigued (fatigue state) in the muscle strength training. FIG. 17 is a similarity matrix of target waveforms in a segment from the terminal stance period to the terminal swing period among time series data of sensor data measured in walking of 200 meters (m). The similarity matrix of FIG. 17 is generated by a procedure similar to that of the similarity matrix of FIG. 16.

In the similarity matrix of FIG. 17, the similarity of the target waveform tends to decrease (darken) as the stride number increases in both normal state (left side) and fatigue state (right side). The area of the dark region in the upper right of the similarity matrix is larger during the fatigue state (right side) than during the normal state (left side). This indicates that the decrease in the similarity of the target waveform starts earlier in the fatigue state (right side) than in the normal state (left side). The decrease in similarity of the target waveform tends to depend on muscle strength such as abduction muscles. It is presumed that the similarity matrix of FIG. 17 reflects a difference in the fatigue level of muscle strength.

The stability evaluation unit 227 evaluates the dynamic stability of gait based on the feature of the similarity matrix. For example, the stability evaluation unit 227 evaluates the dynamic stability of gait according to a change in the feature appearing in the similarity matrix. For example, the stability evaluation unit 227 determines that the dynamic stability of gait decreases for a dark portion of the similarity matrix. For example, the stability evaluation unit 227 evaluates the dynamic stability of gait according to the area of the dark region of the similarity matrix. For example, the stability evaluation unit 227 determines that the dynamic stability of gait is low in a case where the area of the region (dark region) having similarity lower than the reference exceeds a predetermined ratio with respect to the entire area of the similarity matrix. For example, when the area of the dark region with respect to the entire area of the similarity matrix exceeds a predetermined ratio, the stability evaluation unit 227 determines that fatigue is accumulated. For example, the stability evaluation unit 227 estimates the age of the user according to the ratio of the area of the dark region to the entire area of the similarity matrix. For example, the stability evaluation unit 227 may be configured to estimate the age of the user according to the ratio of the area of the dark region to the entire area of the similarity matrix with respect to the sensor data measured at the normal state.

The stability evaluation unit 227 outputs information regarding the dynamic stability of gait. For example, the stability evaluation unit 227 may output a similarity matrix as information regarding the dynamic stability of gait. For example, the information regarding the dynamic stability of gait is output to a display device (not illustrated) or a mobile terminal (not illustrated). The information output to the display device is displayed on a screen of the display device or the mobile terminal. For example, the information regarding the dynamic stability of gait is output to an external system (not shown). The information regarding the dynamic stability of gait can be used for any application. The communication function for outputting the information regarding the dynamic stability of gait is not particularly limited.

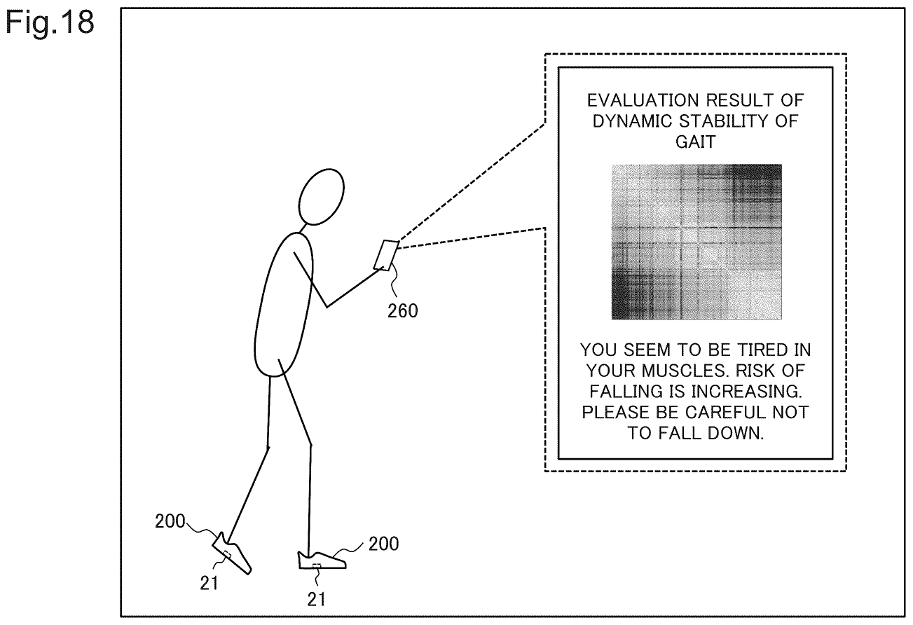

FIG. 18 is a conceptual diagram illustrating an example of a usage scene of the gait measurement system 2. FIG. 18 is an example in which information regarding the evaluation result of the dynamic stability of gait of the user is displayed on the screen of a mobile terminal 260 of the user wearing a shoe 200 on which the measurement device 21 is installed. In the example of FIG. 18, the similarity matrix generated for the user is displayed on the screen of the mobile terminal 160. In the example of FIG. 18, in accordance with the displayed similarity matrix, the information “You seem to be tired in your muscles. The risk of falling is increasing. Please be careful not to fall down.” is displayed in the screen of the mobile terminal 260 as the evaluation result of the dynamic stability of gait. The user who has browsed the information displayed on the screen of the mobile terminal 260 can take an action according to the feature appearing in the similarity matrix or the information relevant to the similarity matrix. For example, the pedestrian who has browsed the information displayed on the screen of the mobile terminal 260 can continue walking while paying attention to falling or take a break while avoiding the risk of falling according to the content of the information.

### (Operation)

Next, an operation of the gait measurement system 2 will be described with reference to the drawings. Description of operations of the measurement device 21 will be omitted. Hereinafter, an example will be described in which a target waveform is generated on the mobile terminal side using sensor data measured by the measurement device 21, and dynamic stability evaluation on gait is performed on the server side based on the target waveform. The following operation of the gait measurement system 2 may include an operation different from the above description of the configuration.

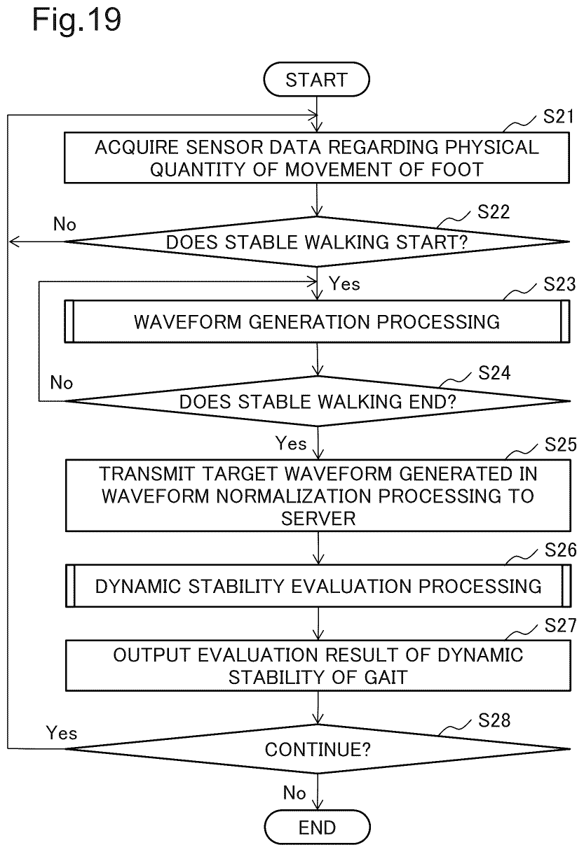

FIG. 19 is a flowchart for explaining an example of the operation of the gait measurement system 2. In the description along the flowchart of FIG. 19, the gait measurement system 2 will be described as an operation subject. In the description along the flowchart of FIG. 19, it is assumed that the matrix generation unit 226 and the stability evaluation unit 227 are arranged on the server side among the configurations included in the gait measurement system 2.

In FIG. 19, the gait measurement system 2 acquires sensor data regarding a physical quantity of the movement of the foot (step S21).

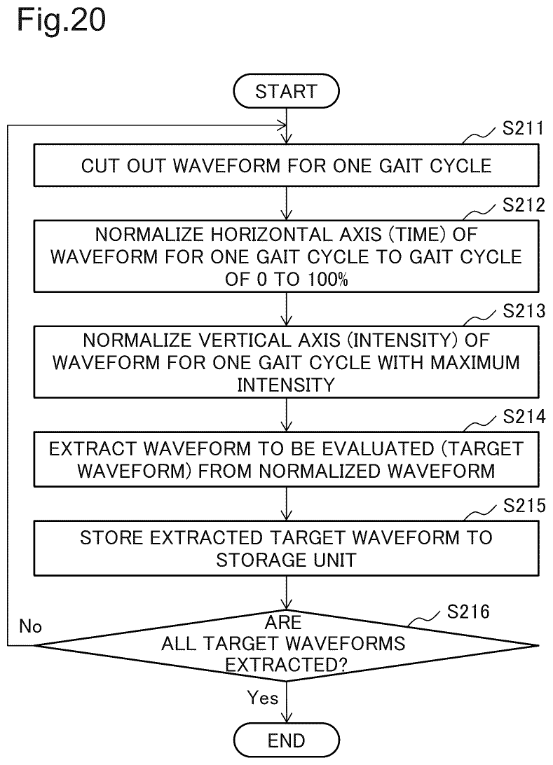

When the start of the walking session (start of stable walking) is detected (Yes in step S22), the gait measurement system 2 executes waveform generation processing (step S23). The waveform generation processing in step S23 will be described later (FIG. 20). When the start of the walking session (start of stable walking) is not detected (No in step S22), the process returns to step S21.

When the end of the walking session (stable walking end) is detected (Yes in step S24), the gait measurement system 2 transmits the target waveform generated by the waveform generation processing to the server (step S25). When the end of the walking session (the end of the stable walking) is not detected (No in step S24), the process returns to step S23.

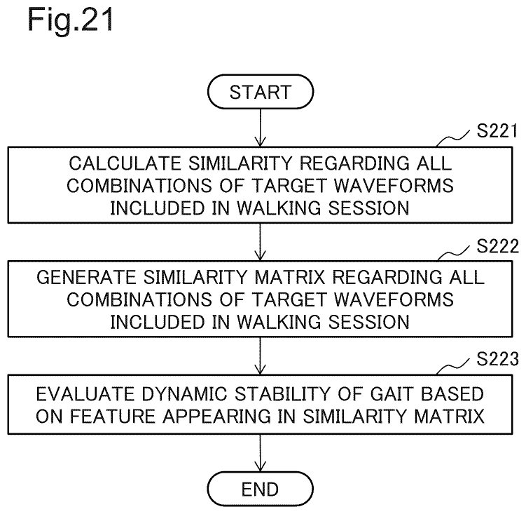

After step S25, the gait measurement system 2 executes the dynamic stability evaluation processing (step S26). Details of the dynamic stability evaluation processing in step S26 will be described later (FIG. 21). In the processing along the flowchart of FIG. 19, the dynamic stability evaluation processing is executed on the server side, but the dynamic stability evaluation processing may be executed on the mobile terminal side. When the dynamic stability evaluation processing is executed on the mobile terminal side, step S25 may be omitted, and step S26 may be executed after step S23.

Next, the stability evaluation unit 227 outputs the evaluation result of the dynamic stability of gait (step S27). After step S27, when the process is continued (Yes in step S28), the process returns to step S21. When the process is not continued (No in step S28), the process according to the flowchart of FIG. 19 is ended. Whether to continue the process may be determined based on a preset criterion.

### [Waveform Generation Processing]

FIG. 20 is a flowchart for explaining the waveform generation processing (step S23 in FIG. 19). In the processing description along the flowchart of FIG. 20, the waveform processing unit 223 included in the gait evaluation device 12 will be described as an operation subject.

In FIG. 20, first, the waveform processing unit 223 cuts out a waveform for one gait cycle from the time series data of the sensor data (step S211).

Next, the waveform processing unit 223 normalizes the time (horizontal axis) of the waveform for one gait cycle to a gait cycle of 0 to 100% (step S212).

Next, the waveform processing unit 223 normalizes the intensity (vertical axis) of the waveform for one gait cycle based on the maximum intensity (step S213). For example, the waveform processing unit 223 normalizes the intensity of the waveform for one gait cycle with the maximum intensity as 1.

Next, the waveform processing unit 223 extracts a waveform to be evaluated for the dynamic stability of gait (also referred to as a target waveform) from the normalized waveform (step S214).

The waveform processing unit 223 stores the extracted target waveform (target waveform) in the storage unit 225 (step S215).

When all the target waveforms have been extracted (Yes in step S216), the process along the flowchart in FIG. 20 ends (the process proceeds to step S24 in FIG. 19). When not all the target waveforms have been extracted (No in step S216), the process returns to step S211.

### [Dynamic Stability Evaluation Processing]

FIG. 21 is a flowchart for explaining the dynamic stability evaluation processing (step S26 in FIG. 19). In the description of the processing along the flowchart of FIG. 21, the matrix generation unit 226 and the stability evaluation unit 227 included in the gait evaluation device 22 will be described as operation subjects.

In FIG. 21, first, the matrix generation unit 226 calculates similarity for all the combinations of target waveforms included in the walking session (step S221).

Next, the matrix generation unit 226 generates a similarity matrix regarding all the combinations of target waveforms included in the walking session (step S222).

Next, the stability evaluation unit 127 evaluates the dynamic stability of gait based on the features appearing in the similarity matrix (step S223). After step S223, the process proceeds to step S27 in FIG. 19.

In the above, the example of calculating the similarity for all the combinations of target waveforms included in the walking session has been described. However, a combination of representative target waveforms may be extracted, and the similarity may be calculated for the combination of extracted target waveforms. For example, a combination of target waveforms of odd-numbered gait cycles may be extracted, or a combination of target waveforms of even-numbered gait cycles may be extracted. For example, a combination of target waveforms for each several gait cycles may be extracted. The extraction of a combination of representative target waveforms is not particularly limited.

As described above, the gait measurement system of the present example embodiment includes the measurement device and the gait evaluation device. The measurement device is disposed on the user's footwear. The measurement device measures the spatial acceleration and the spatial angular velocity according to the gait of the user. The measurement device generates sensor data based on the measured spatial acceleration and spatial angular velocity. The measurement device outputs the generated sensor data to the gait evaluation device. The gait evaluation device includes an identification unit, a waveform processing unit, a matrix generation unit, and a stability evaluation unit. The identification unit identifies a walking session in which stable walking is performed based on the sensor data regarding the movement of the foot. The waveform processing unit extracts a target waveform included in an evaluation target segment of the dynamic stability of gait from time series data of sensor data measured for the same walking session for each gait cycle. The matrix generation unit extracts a plurality of pairs of target waveforms included in the same walking session. The matrix generation unit calculates similarity in a brute-force manner for a plurality of extracted pairs of target waveforms. The matrix generation unit generates a similarity matrix obtained by two-dimensionally mapping a magnitude relationship of similarity among a plurality of pairs of target waveforms. The stability evaluation unit evaluates the dynamic stability of gait based on the features appearing in the similarity matrix.

In the method of the present example embodiment, the transition of the similarity of the target waveform extracted from the evaluation target segment for each gait cycle included in the same walking session can be visualized by the similarity matrix. According to the present example embodiment, the similarity obtained by comparing the target waveform in each gait cycle in a brute-force manner can be reflected in the similarity matrix without omission, so that the dynamic stability of gait can be evaluated more accurately. According to the present example embodiment, the change in the dynamic stability of gait can be more accurately verified based on the transition of the similarity of the target waveform visualized by the similarity matrix.

In one aspect of the present example embodiment, the stability evaluation unit determines that the dynamic stability of gait is low in a case where the area of the region having the similarity lower than the reference in the similarity matrix exceeds a predetermined ratio with respect to the entire area of the similarity matrix. According to this aspect, the dynamic stability of gait can be quantitatively evaluated based on the similarity matrix. According to the present aspect, the fatigue level of the muscles such as the abduction muscles can be verified based on the similarity matrix.

### Third Example Embodiment

Next, a gait evaluation device according to a third example embodiment will be described with reference to the drawings. The gait evaluation device according to the present example embodiment has a configuration in which the gait evaluation devices according to the first and second example embodiments are simplified.

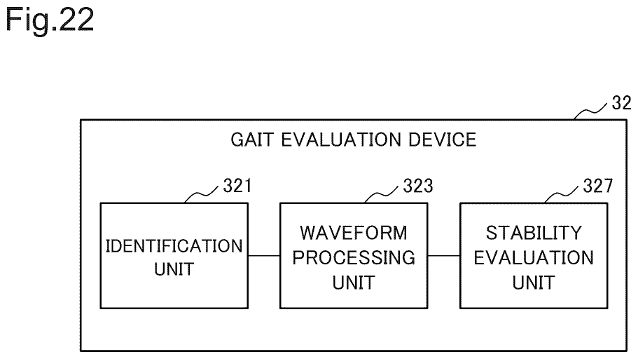

FIG. 22 is a block diagram illustrating an example of a configuration of a gait evaluation device 32 according to the present example embodiment. The gait evaluation device 32 includes an identification unit 321, a waveform processing unit 323, and a stability evaluation unit 327. The identification unit 321 identifies a walking session in which stable walking is performed based on sensor data regarding the movement of the foot. The waveform processing unit 323 extracts a target waveform included in an evaluation target segment of the dynamic stability of gait from time series data of sensor data measured for the same walking session for each gait cycle. The stability evaluation unit 327 evaluates the dynamic stability of gait in response to the transition of the similarity of the target waveform extracted for each gait cycle. The stability evaluation unit 327 outputs an evaluation result of the dynamic stability of gait.

According to the present example embodiment, by evaluating in response to the transition of the similarity of the target waveform extracted from the evaluation target segment for each gait cycle included in the same walking session, it is possible to accurately evaluate the dynamic stability of gait without being affected by an accidental gait variation.

### (Hardware)

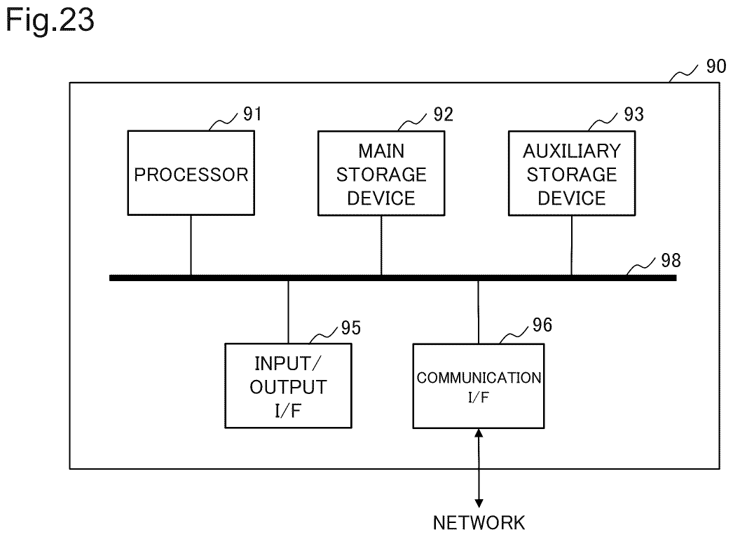

Here, a hardware configuration for executing the processing according to each embodiment of the present disclosure will be described using an information processing device 90 of FIG. 23 as an example. The information processing device 90 in FIG. 23 is a configuration example for executing the processing of each example embodiment, and does not limit the scope of the present disclosure.

As illustrated in FIG. 23, the information processing device 90 includes a processor 91, a main storage device 92, an auxiliary storage device 93, an input/output interface 95, and a communication interface 96. In FIG. 23, the interface is abbreviated as an I/F. The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program stored in the auxiliary storage device 93 or the like in the main storage device 92. The processor 91 executes the program developed in the main storage device 92. In the present example embodiment, a software program installed in the information processing device 90 may be used. The processor 91 executes the processing according to each example embodiment.

The main storage device 92 has a region in which a program is developed. A program stored in the auxiliary storage device 93 or the like is developed in the main storage device 92 by the processor 91. The main storage device 92 is implemented by, for example, a volatile memory such as a dynamic random access memory (DRAM). A nonvolatile memory such as a magnetoresistive random access memory (MRAM) may be configured and added as the main storage device 92.

The auxiliary storage device 93 stores various data such as programs. The auxiliary storage device 93 is implemented by a local disk such as a hard disk or a flash memory. Various data may be stored in the main storage device 92, and the auxiliary storage device 93 may be omitted.

The input/output interface 95 is an interface for connecting the information processing device 90 and a peripheral device. The communication interface 96 is an interface for connecting to an external system or device through a network such as the Internet or an intranet based on a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input information and settings. When the touch panel is used as an input device, the display screen of the display device may also serve as the interface of the input device. Data communication between the processor 91 and the input device may be mediated by the input/output interface 95.

The information processing device 90 may be provided with a display device for displaying information. In a case where a display device is provided, the information processing device 90 may include a display control device (not illustrated) for controlling display of the display device. The display device may be connected to the information processing device 90 via the input/output interface 95.

The information processing device 90 may be provided with a drive device. The drive device mediates reading of data and a program from a recording medium, writing of a processing result of the information processing device 90 to the recording medium, and the like between the processor 91 and the recording medium (program recording medium). The drive device may be connected to the information processing device 90 via the input/output interface 95.

The above is an example of the hardware configuration for enabling the processing according to each example embodiment of the present invention. The hardware configuration of FIG. 23 is an example of a hardware configuration for executing the processing of each example embodiment, and does not limit the scope of the present invention. A program for causing a computer to execute processing according to each example embodiment is also included in the scope of the present invention. Further, a program recording medium in which the program according to each example embodiment is recorded is also included in the scope of the present invention. The recording medium can be achieved by, for example, an optical recording medium such as a compact disc (CD) or a digital versatile disc (DVD). The recording medium may be implemented by a semiconductor recording medium such as a universal serial bus (USB) memory or a secure digital (SD) card. The recording medium may be implemented by a magnetic recording medium such as a flexible disk, or another recording medium. When a program executed by the processor is recorded in a recording medium, the recording medium is associated to a program recording medium.

The components of each example embodiment may be arbitrarily combined. The components of each example embodiment may be implemented by software or may be implemented by a circuit.

Although the present invention has been described with reference to the example embodiments, the present invention is not limited to the above example embodiments. Various modifications that can be understood by those skilled in the art can be made to the configuration and details of the present invention within the scope of the present invention.

### REFERENCE SIGNS LIST

1, 2 gait measurement system
11, 21 measurement device
12,22,32 gait evaluation device
111 acceleration sensor
112 angular velocity sensor
113 control unit
115 data transmission unit
121, 221, 321 identification unit
123, 223, 323 waveform processing unit
125, 225 storage unit
127, 227, 327 stability evaluation unit
226 matrix generation unit

What is claimed is:

1. A gait evaluation device comprising:
a memory storing instructions; and
a processor connected to the memory and configured to execute the instructions to:
identify, based on sensor data regarding movement of a foot, a walking session in which stable walking is performed;
extract, from time series data of the sensor data measured for a same walking session, for each gait cycle, a target waveform which is included in an evaluation target segment of dynamic stability of gait; and
evaluate the dynamic stability of gait in response to a transition of similarity of the target waveform extracted in each gait cycle, and
output an evaluation result of the dynamic stability of gait.

2. The gait evaluation device according to claim 1, wherein
the processor is configured to execute the instructions to
generate a gait waveform in which a gait cycle and intensity are normalized using time series data of the sensor data, and
extract the target waveform included in the evaluation target segment from the generated gait waveform.

3. The gait evaluation device according to claim 1, wherein
the processor is configured to execute the instructions to
evaluate the dynamic stability of gait in response to a transition of the similarity between a reference target waveform in an initial stage of the walking session and a series of the target waveforms included in the walking session.

4. The gait evaluation device according to claim 3, wherein
the processor is configured to execute the instructions to
compare a representative value of the similarity between the reference target waveform and the target waveform in a first stage where a number of steps is less than a predetermined number of steps with a representative value of the similarity between the reference target waveform and the target waveform in a second stage where the number of steps is equal to or greater than the predetermined number of steps, and
evaluate the dynamic stability of gait according to a comparison result between a representative value of the similarity in the first stage and a representative value of the similarity in the second stage.

5. The gait evaluation device according to claim 1, wherein
the processor is configured to execute the instructions to
extract a plurality of pairs of the target waveforms included in a same walking session, calculate the similarity in a brute-force manner for the plurality of extracted pairs of the target waveforms, and generate a similarity matrix obtained by two-dimensionally mapping a magnitude relationship of the similarity for the plurality of pairs of the target waveforms, and
evaluate the dynamic stability of gait based on a feature appearing in the similarity matrix.

6. The gait evaluation device according to claim 5, wherein
the processor is configured to execute the instructions to
determine that the dynamic stability of gait is low in a case where an area of a region having the similarity lower than a reference in the similarity matrix exceeds a predetermined ratio with respect to an entire area of the similarity matrix.

7. The gait evaluation device according to claim 1, wherein
the processor is configured to execute the instructions to
extract the target waveform from the evaluation target segment set according to a type of a muscle of a determination target of a fatigue level,
determine a fatigue level of a muscle of the determination target in response to a long-term transition of the similarity of the target waveform, and
output a determination result of a fatigue level of a muscle of the determination target.

8. A gait measurement system comprising:
a gait evaluation device according to claim 1; and
a measurement device that is disposed on footwear of a user, measures a spatial acceleration and a spatial angular velocity according to walking of the user, generates sensor data based on the measured spatial acceleration and spatial angular velocity, and outputs the generated sensor data to the gait evaluation device.

9. A gait evaluation method causing a computer to execute:
identifying a walking session in which stable walking is performed based on sensor data regarding movement of a foot;
extracting a target waveform included in an evaluation target segment of dynamic stability of gait from time series data of the sensor data measured for a same walking session for each gait cycle;
evaluating the dynamic stability of gait in response to a transition of similarity of the target waveform extracted for each gait cycle; and
outputting an evaluation result of the dynamic stability of gait.

10. A non-transitory recording medium having stored therein a program causing a computer to execute:
a process of identifying, based on sensor data regarding movement of a foot, a walking session in which stable walking is performed;
a process of extracting, from time series data of the sensor data measured for a same walking session, for each gait cycle, a target waveform which is included in an evaluation target segment of dynamic stability of gait;
a process of evaluating the dynamic stability of gait in response to a transition of similarity of the target waveform extracted in each gait cycle; and
a process of outputting an evaluation result of the dynamic stability of gait.
